(19) United States
(12) Patent Application Publication
(10) Pub. No.: US 2010/0009351 A1
(43) Pub. Date: Jan. 14, 2010

(54) Polynucleotide Capture Materials, and Method of Using Same

(75) Inventors: Sundaresh N. Brahmasandra, Ann Arbor, MI (US); Elizabeth Craig, Ypsilanti, MI (US); Kalyan Handique, Ypsilanti, MI (US)

Correspondence Address: FISH & RICHARDSON P.C., PO BOX 1022, MINNEAPOLIS, MN, 55440-1022, US

(73) Assignee: HANDYLAB, INC., Ann Arbor, MI (US)

(21) Appl. No.: 12/172,208

(22) Filed: Jul. 11, 2008

(51) Int. Cl. C12Q 1/68

(52) U.S. Cl. 435/6

(57) ABSTRACT

Methods for processing polynucleotide-containing biological samples, and materials for capturing polynucleotide molecules such as DNA from such samples. The DNA is captured by polyethyeleneimine (PEI) bound to a surface, such as the surface of magnetic particles. The methods and materials have high efficiency of binding DNA and of release, and thereby permit quantitative determinations.

### TECHNICAL FIELD

The technology described herein generally relates to methods for processing biological samples, and more particularly relates to materials for capturing polynucleotide molecules such as DNA from such samples, and permitting quantitative determination thereof.

### BACKGROUND

The analysis of a biological sample such as a clinical sample or a test sample of food, for presence of a pathogen or to determine the presence of a particular gene for example, will typically include detecting one or more polynucleotides present in the sample. One type of detection is qualitative detection, which relates to a determination of the presence or absence of a target polynucleotide and/or the determination of information related to, for example, the type, size, presence or absence of mutations, and/or the sequence of the target polynucleotide. Another type of detection is quantitative detection, which relates to a determination of the amount of a particular polynucleotide present in the sample, expressed for example as a concentration or as an absolute amount by weight or volume. Detection may also include both qualitative and quantitative aspects. Quantitative detection is typically, however, a more challenging pursuit than is a simple qualitative determination of presence or absence of a polynucleotide.

Detecting polynucleotides often involves the use of an enzyme. For example, some detection methods include polynucleotide amplification by polymerase chain reaction (PCR) or a related amplification technique. Other detection methods that do not amplify the polynucleotide to be detected also make use of enzymes. However, the functioning of enzymes used in such techniques may be inhibited by the presence of materials (known as inhibitors) that accompany the polynucleotide in many biological—particularly clinical—samples. The inhibitors may interfere with, for example, the efficiency and/or the specificity of the enzymes.

Polynucleotide detection today is moving towards ever more rapid, and ever more sensitive techniques. However, the application of nucleic acid testing to routine diagnosis of pathogens has been limited to large clinical reference labs and major hospital labs due to the high cost, complexity and skill level requirements for implementing such testing. With the current demands on practice of medicine, laboratories that carry out diagnostic testing on patient samples see substantial benefits from having extremely high throughput, which in itself is assisted if the time to arrive at a diagnostic outcome for a given sample is made as short as possible. Testing may also be made more rapid if the actual sample on which the tests are run is made as small as possible. More recently, there has been a growing need for a small, easy to use, low-cost, automated platform for the extraction of high quality DNA from microorganisms in clinical specimens.

Correspondingly, then, the need to be able to isolate minute quantities of polynucleotides from complex biological samples in a manner that effectively avoids the presence of, or reduces the detrimental impact of, inhibitors is ever more important. Furthermore, given the availability of various stand-alone automated amplification apparatuses, it is desirable to be able to routinely and reliably extract from a raw clinical sample a quantity of polynucleotide that is ready—in terms of purity and quantity—for amplification.

The discussion of the background herein is included to explain the context of the technology. This is not to be taken as an admission that any of the material referred to was published, known, or part of the common general knowledge as at the priority date of any of the claims found appended hereto.

Throughout the description and claims of the specification the word “comprise” and variations thereof, such as “comprising” and “comprises”, is not intended to exclude other additives, components, integers or steps.

### SUMMARY

The technology herein provides excellent DNA capture and recovery via use of micro-particles having a high DNA binding capacity, such as 100 μg/mg beads, and a>90% release efficiency. In exemplary embodiments, 8-10 μg DNA can be extracted from an overnight culture, and 2-4 μg DNA can be obtained from a buccal swab. Processes, as described herein, permit very fast (15-20 minutes including lysis) DNA extraction from cellular material, via a single tube process. Processes, as described herein, comprise a streamlined procedure having fewer steps (such as six) to proceed from raw sample to purified DNA. Such processes therefore provide an extremely effective clean-up of DNA from raw biological samples, thereby permitting PCR to be performed thereon. The methods and processes are applicable across a wide variety of sample matrices, as well as clinical buffers used when collecting raw samples, e.g., M4, UTM, and Todd Hewit Broth.

Suitable targets, that have assays used in clinical testing, and that may be the subject of sample preparation processes as described herein, include, but are not limited to: Chlamydia Trachomatis (CT); Neisseria Gonorrhea (GC); Group B Streptococcus; HSV; HSV Typing; CMV; Influenza A & B; MRSA; RSV; TB; Trichomonas; Adenovirus; Bordatella; BK; JC; HHV6; EBV; Enterovirus; and M. pneumoniae.

One aspect of the present invention relates to a method for processing one or more DNA compounds (e.g., to concentrate the DNA compound(s) and/or to separate the DNA compound(s) from inhibitor compounds (e.g., hemoglobin, peptides, faecal compounds, humic acids, mucousol compounds, DNA binding proteins, or a saccharide) that might inhibit detection and/or amplification of the DNA compounds).

In some embodiments, the method includes contacting the sample containing the DNA compounds and polyethyleneimine (PEI) that preferentially associates with (e.g., retains) the DNA compounds as opposed to inhibitors. The PEI is typically bound to a surface (e.g., a surface of one or more particles). The PEI retains the DNA compounds so that the DNA compounds and inhibitors may be separated, such as by washing the surface with the compound and associated DNA compounds. Upon separation, the association between the DNA compound and compound may be disrupted to release (e.g., separate) the DNA compounds from the compound and surface.

In certain embodiments, more than 90% of a DNA compound present in a sample may be bound to microparticles, prepared by methods herein, released, and recovered.

In certain embodiments, DNA may be bound tomicro-particles according to methods described herein, released, and recovered, in less than about 10 minutes (e.g., less than about 7.5 minutes, less than about 5 minutes, or less than about 3 minutes).

The present disclosure provides for a method of isolating DNA from a cell-containing sample, the method comprising: contacting the sample with a lysis solution and a plurality of binding particles coated in polyethyleneimine, so that the DNA is liberated from the cells and becomes reversibly bound to the polyethyleneimine, thereby creating binding particles bound with DNA and a solution containing residual cellular matter; compacting the binding particles bound with DNA; removing the solution containing residual cellular matter; washing the binding particles; and releasing the DNA from the binding particles.

The present disclosure includes a process for a method of extracting a DNA from a cell-containing sample, the method comprising: contacting the sample with a polyethyeleneimine-bound retention member and a solution of reagents for cell lysis, DNAase activity, and digestion of proteins and lipids; heating the sample, retention member, and reagent solution to 50-60° C. for 7 to 15 minutes; capturing the retention member by a magnet; washing the retention member using 50-100 μl of a buffer comprising 20 mM Tris-EDTA with 1 mM EDTA and 1% Triton X-100 at pH 8.0; replacing the buffer with 10 μl of Tris at pH 8.0 and 1 μl of 20 mM NaOH; heating the retention member at 85° C. for 3-5 minutes; and collecting a supernatant containing DNA.

The present disclosure further includes a process for concentrating DNA from a sample containing polymerase chain reaction inhibitors, the method comprising: contacting between 500 μl and 1 ml of the sample with a plurality of DNA binding particles, the binding particles configured to preferentially retain the DNA in the sample as compared to the polymerase chain reaction inhibitors; concentrating the plurality of particles having the one or more polynucleotides bound thereto into an effective volume between 50 nanoliters and 1 microliters; and releasing the one or more polynucleotides into 3 μl of solution.

The present disclosure still further includes a composition comprising: carboxyl acid group modified microparticles; and polyethyeleneimine bound via one or more amine groups per molecule to one or more of the carboxylic acid groups on the microparticles.

The present disclosure additionally includes a kit, comprising: a number of sealed tubes, each containing lysis buffer; a tube containing lyophilized microparticles having polyethyeleneimine bound thereto; a tube containing liquid wash reagents, sufficient to analyze the number of samples; a tube containing liquid neutralization reagents, sufficient to analyze the number of samples; and a tube containing liquid release reagents, sufficient to analyze the number of samples, wherein each component of the kit is stored in an air-tight container.

The present disclosure further includes a kit, comprising: a first air-tight pouch enclosing a number of tubes, each tube containing lyophilized microparticles having polyethyeleneimine bound thereto; a second air-tight pouch enclosing a number of reagent holders, each holder comprising: a tube containing liquid lysis reagents; a tube containing liquid wash reagents; a tube containing liquid neutralization reagents; and a tube containing liquid release reagents.

The present disclosure still further includes a method of making a polynucleotide retention member, the method comprising: washing a quantity of microspheres with carbonate and MES buffer; preparing sulfo-NHS and EDAC; incubating the microspheres with sulfo-NHS and EDAC for 30 minutes; washing the microspheres with MES and borate buffer; contacting the microspheres with PEI for 8-10 hours; and rinsing unbound PEI from the microspheres.

### BRIEF DESCRIPTION OF THE DRAWINGS

FIG. 1 shows schematically a typical process as described herein.

FIG. 2 shows schematically the action of DNA affinity beads as further described herein.

FIG. 3 shows the use of DNA extraction reagents and process, as described herein, to isolate and purify Gonorrheoea cells in urine.

FIG. 4 shows the use of DNA extraction Reagents and process, as described herein, to isolate and purify Group B Streptococcus cells.

FIG. 5 shows the use of DNA extraction reagents and process, as described herein, to isolate and purify S. aureus cells in whole blood.

FIG. 6 shows illustrates the analytical sensitivity of the process.

FIG. 7 shows illustrates the analytical sensitivity of the process.

FIG. 8 shows quantitation of DNA detection in urine.

FIG. 9 shows quantitation of DNA detection in plasma.

FIG. 10 shows a flow chart of a way of making polyethyleneimine coated micro-particles.

Like reference symbols in the various drawings indicate like elements.

### DETAILED DESCRIPTION

Analysis of biological samples often includes determining whether one or more polynucleotides (e.g., a DNA, RNA, mRNA, or rRNA) is present in the sample. The technology described herein has greatest applicability to determining the DNA that is present in a sample. For example, a sample may be analyzed to determine whether the DNA of a particular pathogen is present. If present, the DNA may be indicative of a corresponding disease or condition.

Accordingly, the technology described herein is directed to materials that bind DNA, and use of such materials in isolating DNA from biological samples. The materials, in conjunction with methods of using the materials, provide for rapid and reliable extraction of DNA from many different types of biological samples, including quantitative determination of the DNA. Such methods are typically referred to as “sample preparation” methods. What is meant by such a term is the liberation, extraction, concentration, and/or isolation, of DNA of a target organism from a raw sample—such as obtained directly from a patient or an agricultural or food product—where the raw sample contains the target DNA bound in cellular form. The liberated target DNA is placed, at the culmination of the process, in a form suitable for amplification and/or detection.

The term DNA (deoxyribonucleic acid) as used herein can mean an individual molecule or population of molecules, such as identifiable by having a specific nucleotide sequence common to all, or can mean collectively molecules of DNA having different sequences from one another. For example, a biological sample from a human patient may contain DNA from the patient's cells, having one sequence, and DNA from cells of a pathogen, having a different sequence. The sample is thus referred to as containing DNA, even though there are molecules of DNA in the sample that are different (chemically distinct) from one another. The methods herein can be used to liberate, collectively, molecules of DNA from both the patient's and the pathogen's cells in such a sample. Typically, however, in such an instance, it will be the DNA of the pathogen that will be of interest and which will be selectively amplified from amongst all the DNA that is ultimately isolated from the sample. The DNA that is best suited for extraction by the methods herein has a size less than 7.5 Mbp, though it would be understood that larger DNA molecules may be susceptible to extraction and detection by the methods herein.

Typically, biological samples are complex mixtures. For example, a sample may be provided as a blood sample, a tissue sample (e.g., a swab of, for example, nasal, buccal, anal, or vaginal tissue), a biopsy aspirate, a lysate, as fungi, or as bacteria. The DNA to be determined is normally contained within particles (e.g., cells such as white blood cells, or red blood cells), tissue fragments, bacteria (e.g., gram positive bacteria, or gram negative bacteria), fungi, or spores. One or more liquids (e.g., water, a buffer, blood, blood plasma, saliva, urine, cerebral spinal fluid (CSF), or organic solvent) is typically part of the sample and/or is added to the sample during a processing step. The materials and methods described herein are compatible with a variety of clinical matrices, at least including blood, urine, CSF, swab, plasma.

Methods for analyzing biological samples include releasing DNA from the particles (e.g., bacteria) in the sample, amplifying one or more of the released DNA (e.g., by polymerase chain reaction (PCR)), and determining the presence (or absence) of the amplified polynucleotide(s) (e.g., by fluorescence detection).

Clinical samples present a variety of challenges especially in the detection of target polynucleotides through PCR or similar technologies. A target nucleic acid could be present in a concentration as low as 10 copies per milliliter as measured against a background of millions or billions of copies of competing nucleic acids (such as from a patient's normal cells). Moreover, a variety of other biochemical entities present in the clinical sample inhibit PCR. The inhibitors may also frustrate isolation of the DNA from the sample, such as by being captured by a material designed to retain the DNA. If the concentration of inhibitors is not reduced relative to the DNA to be determined, the analysis can produce false negative results. Examples of these inhibitors, dependent upon the biological sample in question, are cellular debris such as membrane fragments, humic acids, mucousal compounds, hemoglobin, other proteins such as DNA binding proteins, salts, DNAases, fecal matter, meconium, urea, amniotic fluid, blood, lipids, saccharides, and polysaccharides. For example, such inhibitors can reduce the amplification efficiency of DNA by PCR and other enzymatic techniques for determining the presence of DNA.

Therefore, an effective sample preparation method should lead to a concentration of the target DNA and should minimize presence of inhibitory substances. The methods described herein may increase the concentration of the DNA to be determined and/or reduce the concentration of inhibitors relative to the concentration of DNA to be determined.

In addition, cells of some target organisms, such as gram positive bacteria (e.g. Group B Streptococcus), are very hard to lyse, meaning that lysing conditions can be very severe. Such organisms may require additional chemicals for lysing, such as mutanolysin, and may also require higher temperatures for optimal lysis. Such conditions may be accommodated by the materials and methods described herein.

### Sample Preparation Process

A typical sample preparation process may be carried out in a processing chamber that includes a plurality of particles (e.g., beads, microspheres) configured to retain DNA of the sample under a first set of conditions (e.g., a first temperature and/or first pH) and to release the DNA under a second set of conditions (e.g., a second, higher temperature and/or a second, more basic, pH). Typically, the DNA is retained preferentially as compared to inhibitors that may be present in the sample.

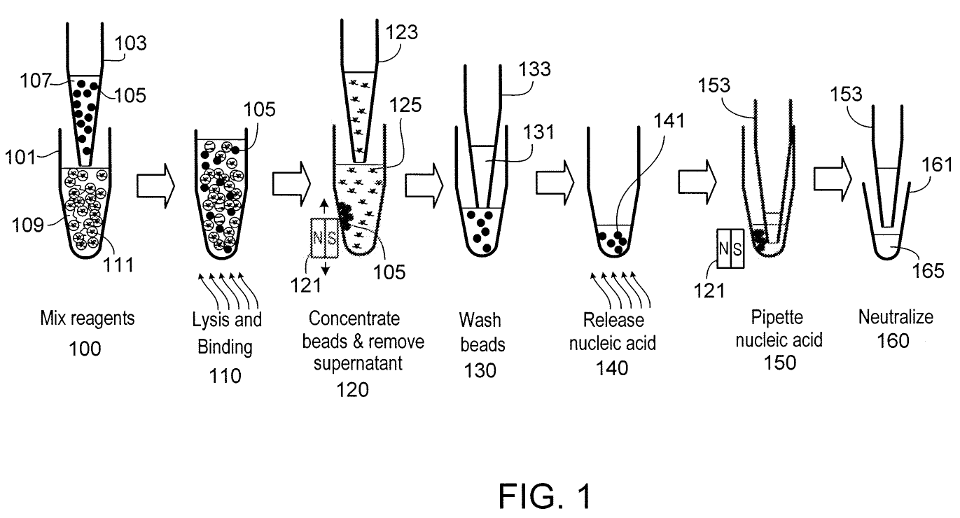

An exemplary sample preparation process is illustrated in FIG. 1. The various reagents referred to in connection with FIG. 1 are described in further detail elsewhere herein. At 100, a process tube 101, such as a standard laboratory 1.7 ml microcentrifuge tube, contains a biological sample comprising a liquid 109, such as an aqueous solution, and cellular materials 111, wherein at least some of the cellular materials may contain DNA of a target of interest. The biological sample may be any of those described elsewhere herein, and process tube 101 may be any tube or suitable vessel, as further described herein. It is to be understood that, although the process is illustrated with respect to FIG. 1, the process is not limited to be carried out in a tube. The sample and various reagents may be, for example, delivered to, and mixed and reacted within, chambers of a microfluidic device such as a microfluidic cartridge, as further described in U.S. application Ser. No. 11/281,247, filed Nov. 16, 2005 and incorporated herein by reference.

A first pipette tip 103 contains a solution 107 of microparticles 105, that are delivered to the process tube and contacted with the biological sample contained therein. The surfaces of particles 105 are modified to have PEI attached, as further described herein, so that they retain DNA in preference to inhibitors in solution. Solution 107 may be a lysis solution, as further described herein. The lysis solution may contain a detergent, in addition to various enzymes, as described elsewhere herein. Thorough mixing of the microparticles, the solution, and the biological sample may occur simply by turbulent combination of the two solutions upon release of the microparticle containing solution from the pipette tip, or may occur via mechanical or manual agitation of process tube 101.

First pipette tip 103 is positioned above process chamber 101, such as by manual operation by a user, or such as by an automated pipetting head, an example of which is described in U.S. provisional patent application Ser. No. 60/959,437, filed Jul. 13, 2007 which is incorporated herein by reference.

At 110, using the same process tube 101, the microparticles, biological sample, and lysis reagents are incubated, such as by applying heat from an external source, as shown, so that the cells in the biological sample are lysed, and liberate DNA. Under these conditions, the DNA molecules also bind to suitably configured surfaces of the micro-particles, as further described herein. Typically, the particles retain DNA from liquids having a pH about 9.5 or less (e.g., about 9.0 or less, about 8.75 or less, about 8.5 or less). It is to be noted that the binding of DNA to the affinity microparticles happens concurrently with the lysis process, and the binding is not adversely affected by the presence of detergents and, in some instances, lytic enzymes in the lysis solution. The choice of temperature is dictated by what is required to lyse the cells in question, and heat is not required to effectuate binding of the DNA to the particles. Typically, those cells having tougher cell walls (e.g., lysteria, or anthrax) will require higher temperatures. For example, Chlamydia determination utilizes a temperature of 37° C. for a duration of 5-10 minutes for lysis and binding, whereas Group B Streptococcus determination utilizes a temperature of 60° C. for a duration of 5-10 minutes. Generally, the liquid is heated to a temperature insufficient to boil liquid in the presence of the particles.

At 120, the microparticles are concentrated or compacted, and the remaining solution containing residual cellular matter 125 is removed, for example by a second pipette tip 123. By compacted is meant that the microparticles, instead of being effectively uniformly distributed through a solution, are brought together at a single location in the process tube, in contact with one another. Where the microparticles are magnetic, compaction of the microparticles may be achieved by, for example, bringing a magnet 121 into close proximity to the outside of the process chamber 101, and moving the magnet up and down outside the chamber. The magnetic particles are attracted to the magnet and are drawn towards the inside of the wall of the process chamber adjacent the magnet.

Pipette tip 123 removes as much of the remaining solution (sometimes referred to as supernatant, or solution having residual cellular matter) as is practical without drawing up significant quantities of microparticles. Typically a pipette tip may slide into process chamber 105 without contacting the microparticles. In this way the microparticles are concentrated, by being present in a smaller volume of solution than hitherto. Pipette tip 123 may be a different tip from pipette tip 103, or may be the same tip. In some embodiments, after removal of the solution containing residual cellular matter, less than 10 microliters of solution is left along with the particles. Typically this is achieved by both compaction of the microsparticles to a small pellet, but also positioning that pellet away from the location wherein the pipette will be introduced for removal of the supernatant. The positioning of the pipette in relation to the bottom of the tube is also important so that almost all of the supernatant is removed. The pipette tip should be almost close to the bottom of the tube (within 1-2 mm) but without completely sealing the pipette tip against the tube bottom. A stellated pattern may also be used at the bottom of the lysis tube, (as described in U.S. provisional patent application Ser. No. 60/959,437, filed Jul. 13, 2007, and incorporated herein by reference), but the positioning of the patterns in relation to the location of the magnet becomes important so that the sliding of the compacted microparticles is not hindered and the crevices between vertices of the stellated pattern do not trap microparticles.

At 130, a third pipette tip 133 delivers a wash solution 131 to the process chamber 101 containing compacted microparticles. The wash solution may comprise, e.g., a buffer such as Tris-EDTA with a surfactant such as 1% Triton X 100, and having an overall pH 8.0. Typically, the volume of wash buffer is 100 microliters or less, where the sample is 2 ml or less in volume. The wash solution is used to wash off any non-DNA molecules, such as inhibitors, that may have become bound to the microparticles. The wash solution is chosen to preferentially wash off non-DNA molecules while leaving in place those DNA molecules bound to the microparticles. Pipette tip 133 may be a different tip from either or both of pipette tips 103 and 123, or may be one of those tips being re-used.

In order to release the DNA from the particles, the wash solution 131 is replaced with a strong alkaline (pH>12) release solution, e.g., a sodium hydroxide solution, or a buffer solution having a pH different from that of the wash solution. This can be done by pipetting out as much of the wash solution as possible, for example, having a residual volume <5 microliters, and then dispensing release buffer with a new pipette tip. In case the same tip is used, the liquid should be completely drained off so as not to dilute the release solution. For example, at 140, a release solution 141 is delivered to process chamber 101 so that the DNA bound to the micro-particles can be liberated from those micro-particles. In general, the PEI on the particles most efficiently release DNA when the pH is about 12 or greater. Consequently, DNA can be released from the particles into the surrounding liquid. In some instances, heat may be applied to the process tube, such as to heat the solution to 85° C., to facilitate release of the DNA. Generally, the liquid is heated to a temperature insufficient to boil liquid in the presence of the particles. In some embodiments, the temperature is 100° C. or less (e.g., less than 100° C., about 97° C. or less). In some embodiments, the temperature is about 65° C. or more (e.g., about 75° C. or more, about 80° C. or more, about 90° C. or more). In some embodiments, the temperature is maintained for about 1 minute or more (e.g., about 2 minutes or more, about 5 minutes or more, about 10 minutes or more). In some embodiments, the temperature is maintained for about 30 minutes (e.g., about 15 minutes or less, about 10 minutes or less, about 5 minutes or less). In some embodiments, the process tube is heated to between about 65 and 90° C. (e.g., to about 70° C.) for between about 1 and 7 minutes (e.g., for about 2 minutes). In other embodiments, the heating is to 85° C. for 3 minutes. In still other embodiments, the heating is to 65° C. for 6 minutes. In general, a longer heating time is required for a lower temperature. Alternatively, or in combination, particles with retained DNA are heated to release the DNA without assistance of a release solution. When heat alone is used to release the DNA, the release solution may be identical with the wash solution.

Typically, the DNA from a 2 ml sample, and according to the description of the lysis, binding, and washing described elsewhere therein, is released into about 20 microliters or less (e.g., about 10 microliters or less, about 5 microliters or less, or about 2.5 microliters or less) of liquid.

While releasing the DNA has been described as including heating, the DNA may be released without heating. For example, in some embodiments, the release solution has an ionic strength, pH, surfactant concentration, composition, or combination thereof that releases the DNA from the retention member without requiring heat.

It is to be noted that excessive shearing, such as is caused by rapid movements of the liquid during suck-and-dispense mixing operations during wash and release (typically during DNA release) in the sample preparation process may release PEI from the surface of the particles, which itself causes downstream inhibition of PCR. The mixing steps should be limited to less than 10 suck-and-dispense operations, where the amount moved back and forth ranges from 1-20 microliters moved in the pipette, performed over 1-10 seconds per suck-and-dispense operations.

At 150 the microparticles, now having essentially no DNA bound thereto, can be compacted or concentrated in a similar manner to that described for 120, but in this case to facilitate removal of the release solution containing the RNA dissolved therein. For example, magnetic beads can be collected together on the interior of the process chamber wall by bringing magnet 121 into close proximity to the outside of the process chamber. In FIG. 1, magnet 121 is used to compact the microparticles in both 120 and 150, though it would be understood that a different magnet could be used in both instances.

It is to be noted that, thus far, all of the processing steps have taken place in a single tube. This is advantageous for a number of reasons: first, that unnecessary liquid transfer steps will necessarily lead to some loss of target material. Additional liquid transfer steps will also add to the overall time of the protocol. It should be noted that performing all the liquid processing in a single tube is not an easy task primarily because of the residual volumes left between successive liquid transfers. It becomes even more difficult when the final elution volume is very low, such as less than 30 microliters, or less than 20 microliters or less than 10 microliters, or less than 5 microliters. Nevertheless, with the protocols described herein, very good yields may be obtained.

The DNA liberated from the microparticles can be drawn up into a fourth pipette tip 153 in solution in the release solution. Pipette tip 153 need not be different from all of pipette tips 103, 123, and 133 and may therefore represent a re-use of one of those tips. Although it is desirable to use magnetic beads, non-magnetic beads may also be used herein, and separated by, e.g., centrifugation, rather than by use of a magnet.

In certain embodiments, the ratio of the volume of original sample introduced into the processing tube to the volume of liquid into which the DNA is released is at least about 10 (e.g., at least about 50, at least about 100, at least about 250, at least about 500, at least about 1,000). In some embodiments, DNA from a sample having a volume of about 2 ml can be retained within the processing tube, and released, after binding and washing, into about 4 microliters or less (e.g., about 3 microliters or less, about 2 microliters or less, about 1 microliter or less) of liquid.

In some embodiments, the sample has a volume larger than the concentrated volume of the binding particles having the DNA bound thereto by a factor of at least about 10.

In other embodiments, the sample has a volume of 100 μl-1 ml, and the compacted particles occupy an effective volume of less than 2 microliters.

The liquid into which the DNA is released typically includes at least about 50% (e.g., at least about 75%, at least about 85%, at least about 90%, or at least about 95%) of the DNA present in the sample 109. Thus, for example, ˜8-10 μg DNA can be liberated from 1 ml of overnight culture, and ˜2-4 μg DNA can be extracted from one buccal swab. The concentration of DNA present in the release liquid may be higher than in the original sample because the volume of release liquid is typically less than the volume of the original liquid sample. For example, the concentration of DNA in the release liquid may be at least about 10 times greater (e.g., at least about 25 times greater, at least about 100 times greater) than the concentration of DNA in the sample 109. The concentration of inhibitors present in the liquid into which the DNA is released is generally less than the concentration of inhibitors in the original fluidic sample by an amount sufficient to increase the amplification efficiency for the DNA over that which could be obtained from an unpurified sample.

In general, although the processes and materials described herein are capable of performing well—usually with only routine adaptation—over a wide range of sample sizes, and reagent volumes, for most practical applications (considering the size of most biological samples subject to diagnostic analysis), the volume of compacted particles having DNA bound thereto that results (prior to release) is in the range 2-3 μl, and is independent of the sample volume, up to about 2 ml of sample. Typically the quantity of microparticles required is determined by the quantity of DNA in the sample. It is found that, given the efficiency of binding to the particles, 0.5 mg of particles is sufficient for most manual applications, and most involving automated pipetting, regardless of sample size. Thus, for example, for samples having volumes from 0.5 microliters to 3 milliliters, the volume of the compacted particles is 2-3 μl. For example, for Chlamydia, the sample size is typically 1 ml, and 0.5 mg of particles is sufficient. For other applications, DNA from a 2 ml sample can also be extracted with 0.5 mg particles, or in some instances 1 mg beads can be used, and an elution volume of 30 μl. For smaller samples, such as having a volume of 50 μl, it is still typical to use only 0.5 mg particles.

In order to agitate the solution at various stages during the manual process, the solution may be pipetted up and down a number of times, such as 10 times, 15 times, or 20 times. Such a procedure is acceptable during the release step as well as the wash steps. Vortexing also works for these steps. However, for the automated process, the number of mixing operations is kept at a minimum as this was possibly causing some PEI to come off and inhibit downstream PCR.

The process described herein represents an extremely effective clean-up of a sample in preparation for PCR and provides the capability to detect as few as 25 copies of DNA from 1 milliliter of clinical sample. The DNA is present in a high level of concentration because the elution volume can be as low as 3 microliters. There is also a low residual sample liquid and/or wash volume in the concentrated microspheres, thereby minimizing dilution by sample or wash buffer, as well as minimizing inhibition from residual sample

The time interval between introducing the DNA containing sample to processing tube 101, and releasing the DNA into the release liquid is usually between 10 and 30 minutes, and is typically about 15-20 minutes, or may be 15 minutes or less (e.g., about 10 minutes or less, about 5 minutes or less). These times include the lysis time (which doubles up as a sample-binding time), and are extremely fast.

Optionally, at 160 in FIG. 1, the released DNA in solution may be neutralized by contacting it with a neutralization solution 165 (e.g., an equal volume of 25-50 mM Tris-HCl buffer pH 8.0). For example, the DNA in solution in pipette tip 153 may be released into a second process chamber, or vessel, 161 such as a standard laboratory PCR tube, in which the neutralization solution is present. The PCR tube may be removed and introduced into a PCR machine for further analysis.

The DNA in solution in vessel 161 is in a state that it can be amplified, such as by PCR, and detected. Furthermore, the foregoing process steps are extremely reliable and robust, and enable quantitative assays of the extracted DNA over 7 log dilutions (10-10⁷ copies of target DNA/ml of sample).

The process of FIG. 1 has demonstrated effectiveness in manual as well as automated formats.

The process shown in FIG. 1 may be carried out in conjunction with a reagent holder, in which the process chamber may be situated, and in which are found appropriate quantities of microparticles, lysis solution, wash solution, release solution, and neutralization solution, each of which is accessible to one or more pipette tips and for use as shown in FIG. 1. An exemplary reagent holder is described in U.S. provisional patent application Ser. No. 60/959,437, filed Jul. 13, 2007, and in U.S. design patent application Ser. no. 29/______, filed Jul. 11, 2008, (attorney docket no. 19662-085001, entitled “Reagent holder”), all of which are incorporated by reference herein.

Where a magnet is shown in FIG. 1 for use in compacting magnetic microparticles, a magnetic separator, as described in U.S. provisional patent application Ser. No. 60/959,437, filed Jul. 13, 2007 incorporated by reference herein, may be used.

Where it is shown in FIG. 1 that heat may be applied to process chamber 101, a heater assembly, as described in U.S. provisional patent application Ser. No. 60/959,437, filed Jul. 13, 2007, incorporated by reference herein, may be used.

The process shown in FIG. 1 is optimally used to prepare highly pure and concentrated DNA for use in low-volume (e.g. 4 μl) PCR reactions, such as may be carried out in a microfluidic cartridge, for example a microfluidic cartridge described in U.S. provisional patent application Ser. No. 60/959,437, filed Jul. 13, 2007, and incorporated herein by reference.

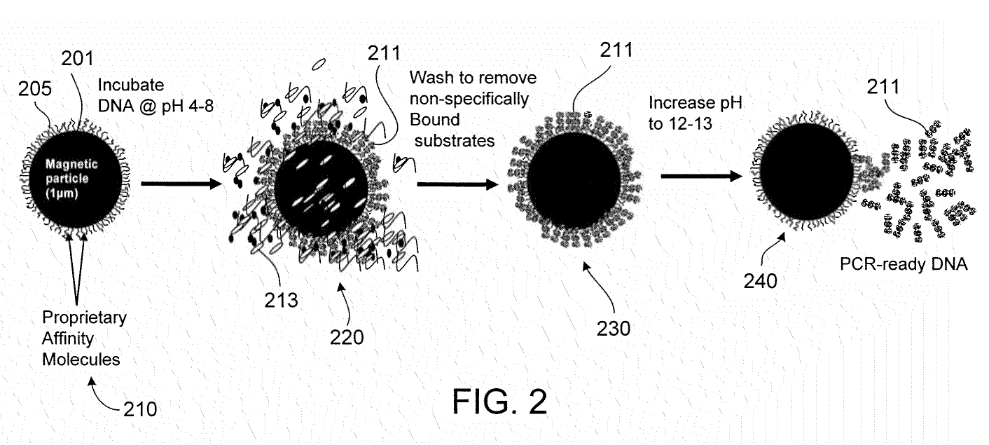

FIG. 2 shows, schematically, a sample preparation process at the molecular level. At 210, a typical magnetic particle 201, having a diameter of 1 μm, is shown. Attached to the surface of particle 201 are molecules 205 having a binding affinity for polynucleotides in solution surrounding the particle. Attachment of molecules 205 is usually via covalent bonds. Such molecules are further described herein and in some embodiments are molecules of polyethyleneimine. From 210 to 220, the magnetic particle is incubated in a solution containing DNA, at a pH of 4-8, lower than the pKₐ of molecules 205. At 220, particle 201 is shown having DNA molecules 211 attached to the affinity molecules 205. Also shown are various other non-specifically bound substrates 213, denoted by small ovals, cigar-shapes, and curved lines.

Moving from 220 to 230 in FIG. 2, the particle 201, to which is bound both DNA molecules 211 and non-specifically bound molecules 213, is washed to remove the non-specifically bound substrates, leaving a particle coated in affinity molecules 205 and DNA molecules 211 bound thereto. From 230 to 240, the DNA molecules 211 are released from the surface of the particle by increasing the pH of the solution surrounding the particle to a pH of 12-13. The released DNA molecules can be collected in a PCR-ready format.

While samples and various solutions have been described herein as having microliter scale volumes, other volumes can be used. For example, processing tubes with surfaces (e.g., particles) configured to preferentially retain DNA as opposed to inhibitors may have large volumes (e.g., many tens of microliters or more, at least about 1 milliliter or more). In some embodiments, the processing tube has a bench-top scale, and other solutions are correspondingly scaled up.

### DNA Capture Material

Suitable DNA affinity molecules are those that offer a very high density of positively ionizable charges at a low pH, and enable strong attraction and binding of polynucleotides including DNA from a clinical lysate within a few minutes.

A typical embodiment of the materials herein uses polyethyleneimine (PEI) as the affinity molecule to bind DNA from a solution. Polyethyleneimine (PEI) is a polymer whose molecules are built from repeating units of ethyleneimine (also known as aziridine) whose three-membered rings open during polymerization.

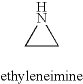

An exemplary PEI molecule has formula as follows.

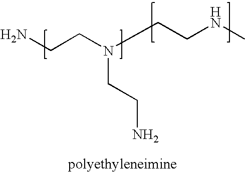

PEI is thus typically a branched molecule built up of units of ethyleneimine that bond through a nitrogen atom.

In certain embodiments, the form of PEI used is a product purchased from the Sigma-Aldrich Chemical Company (“Sigma-Aldrich”), product number 408719 (100 ml). This molecule is ethylenediamine end-capped polyethyleneimine, having (according to Sigma-Aldrich) an average molecular weight of ˜800 when measured by light scattering (LS), and an average molecular weight of ˜600 when measured by gel permeation chromatography (GPC). Ethyleneimine (CH₂CH₂NH) has a molecular weight of 43 and, assuming the average molecular weight of the PEI molecule to be 800 as reported by Sigma-Aldrich, this molecule has approximately 18 units of CH₂CH₂NH, some of which may be branched. In essence it has approximately 18 primary (—NH₂), secondary (—NHR) or tertiary (—NR₂) amine groups present (wherein R is a carbon-containing group bonding to the amine nitrogen through a carbon atom).

The form of PEI suitable for use herein is not limited to that product available from Sigma-Aldrich, however. PEI, being polymeric in nature, admits of a wide range of forms, controlled at least in part by the extent of polymerization permitted during its synthesis. Thus, many variants of PEI, having variously, different numbers of repeating units, and different amounts of branching, are suitable for use herein. For example, one having from 10-30 units of CH₂CH₂NH is suitable, as is one having from 12-24 units, as are those having from 16-20 units. In general, there is a range of lengths that is suitable for polynucleotide capture: smaller lengths don't capture enough DNA, whereas longer lengths retain the DNA too strongly and do not permit easy release. Additionally, different end-caps from ethylene diamine may be used to make a variant of PEI suitable for use herein. Such end caps may include, without limitation, 1,2-propylene diamine, 1,3-propylene diamine, 1,2-butylene diamine, 1,3-butylene diamine, and 1,4-butylenediamine.

Molecules of PEI suitable for use herein may also be characterized by molecular weight. In particular, suitable PEI molecules have weights in the range 600-800 Da. When measured by LS, suitable PEI molecules have measured weights in the range 700-900 Da, and when measured by GPC, suitable PEI molecules have measured weights in the range 500-700 Da.

PEI can itself function as an inhibitor of enzymatic processes such as DNA amplification and therefore it is important that it be used in a manner in which it does not reside in solution together with DNA. Aspects of this are further described in the Examples, hereinbelow.

### Support Materials

During use, PEI is typically immobilized on, such as bound to the surface of, a solid support such as carboxylated beads, or magnetic or non-magnetic beads. In many embodiments, such a solid support comprises microparticles, such as beads, and microspheres. These terms, microparticles, beads, and microspheres may be used interchangeably herein. The particles are typically formed of a material to which the PEI can be easily associated. Exemplary materials from which such particles can be formed include polymeric materials that can be modified to attach a ligand. Typically, such a solid support itself may be derivatized to yield surface functional groups that react easily with PEI molecules to create a chemical bond between the surface and the PEI. A frequently-employed—and desirable—surface functional group is the carboxylic acid (—COOH) group. Exemplary polymeric materials that provide, or can be modified to provide, carboxylic groups and/or amino groups available to attach PEI include, for example, polystyrene, latex polymers (e.g., polycarboxylate coated latex), polyacrylamide, polyethylene oxide, and derivatives thereof. Polymeric materials that can used to form suitable particles are described in U.S. Pat. No. 6,235,313 to Mathiowitz et al., which patent is incorporated herein by reference. Other materials include glass, silica, agarose, and amino-propyl-tri-ethoxy-silane (APES) modified materials.

During the process of reaction of a PEI molecule with a carboxylated particle, such as a magnetic particle, one of the amine groups out of the total possible amine groups on a PEI molecule, such as 18 possible groups in the aforementioned product from Sigma Aldrich, is consumed to react with the COOH group of the surface of the particle to form a carbodiimide bond. (See, e.g., U.S. application #11/281,247, page 40). The remainder of the total number amine groups, such as 17 groups in the aforementioned product from Sigma Aldrich, are available for protonation.

In some embodiments, a synthetic protocol comprises: washing a quantity of microspheres with carbonate and MES buffer; preparing sulfo-NHS and EDAC; incubating the microspheres with sulfo-NHS and EDAC for 30 minutes; washing the microspheres with MES and borate buffer; contacting the microspheres with PEI for 8-10 hours; and rinsing unbound PEI from the microspheres. An example of synthetic protocols for making PEI-bound microparticles, is given in the Examples, hereinbelow.

There are a variety of sources of bead or particle that can be used to bind PEI, and used in the processes described herein, for example: Seradyn Magnetic carboxyl modified magnetic beads (Part #3008050250, Seradyn), Polysciences BioMag carboxyl beads, Dynal polymer encapsulated magnetic beads with a carboxyl coating, and Polybead carboxylate modified microspheres available from Polyscience, catalog no. 09850.

The high density of the PEI molecules on bead surfaces permits even a small quantity of beads (0.5 mg) to be used for clinical samples as large as a milliliter, and permits binding of even low levels of target DNA (<100 copies) in a background of billions of copies of other polynucleotides.

In some embodiments, at least some (e.g., all) of the particles are magnetic. In alternative embodiments, few (e.g., none) of the particles are magnetic. Magnetic particles are advantageous because centrifugation is generally not required to separate them from a solution in which they are suspended.

Particles typically have an average diameter of about 20 microns or less (e.g., about 15 microns or less, about 10 microns or less). In some embodiments, particles have an average diameter of at least about 4 microns (e.g., at least about 6 microns, at least about 8 microns). Magnetic particles, as used herein, typically have an average diameter of between about 0.5 microns and about 3 microns. Non-magnetic particles, as used herein, typically have an average diameter of between about 0.5 microns and about 10 microns.

The particle density is typically at least about 10⁷ particles per milliliter (e.g., about 10⁸ or about 10⁹ particles per milliliter). For example, a processing region, such as present in a microfluidic device configured for used in sample preparation, with a total volume of about 1 microliter, may include about 10³ beads.

In some embodiments, at least some (e.g., all) the particles are solid. In some embodiments, at least some (e.g., all) the particles are porous (e.g., the particles may have channels extending at least partially within them).

The microparticles described herein are not only suitable for use in process tubes that are handled by manual pipetting operations, but they can be used in a microfluidic devices, such as in sample concentrator, thereby enabling even sub-microliter elution volumes to be processed, as applicable.

The microparticles having PEI bound thereto are particularly effective at capturing, and releasing DNA. In some embodiments, the ratio by weight of the DNA captured by the binding particles, to the binding particles prior to contact with the DNA, is 5-20%. In other embodiments, the ratio is 7-12%. In still other embodiments, the ratio is about 10%, corresponding to, e.g., 100 μg of DNA for each mg of particles.

The microparticles having PEI bound thereto are particularly effective at capturing DNA consistently over a wide range of concentrations, thereby permitting quantitative analysis of the DNA to be carried out. In some embodiments, the binding particles capture 90% or more of the DNA liberated from cells into a solution in contact with the binding particles, over a range of 1 to 10⁷ copies of target DNA/milliliter of sample.

In some embodiments, the binding particles release 90% or more of the DNA bound thereto when certain release conditions are deployed.

### Sample Preparation Kits

Microparticles, coated with polyethyeleneimine, can be provided to a user in solid form, such as in lyophilized form, or in solution. It is desirable that the reagent, however provided, can be used immediately by a user for whatever intended purpose, without any preparatory steps. Microparticles prepared by the methods described herein can be lyophilized by methods known in the art, and applicable to microparticles of the sizes and characteristics described herein.

Such microparticles can also be provided in kit form, in conjunction with other reagents that are used, for example, in sample preparation. One embodiment of a kit comprises a number of, such as 24, sealed tubes, each containing lysis buffer; a tube containing lyophilized microparticles having polyethyeleneimine bound thereto; a tube containing liquid wash reagents, sufficient to analyze the number of samples; a tube containing liquid neutralization reagents, sufficient to analyze the number of samples; and a tube containing liquid release reagents, sufficient to analyze the number of samples, wherein each component of the kit is stored in an air-tight container. Other numbers of tubes available in kit form include 12, 25, 30, 36, 48, 50, 60, and 100. Still other numbers are also permissible and consistent with the description herein.

Furthermore, in other embodiments of such a kit, the tube containing lyophilized microparticles can additionally contain particles of reagents selected from the group consisting of: proteinase-k; proteinase-k and mutanolysin; and proteinase-k, mutanolysin, and an internal control DNA. The additional enzymes are often used in cell-specific lysis applications.

In other embodiments, a kit comprises: a first air-tight pouch enclosing a number of—such as 24—tubes, each tube containing lyophilized microparticles having polyethyeleneimine bound thereto; a second air-tight pouch enclosing a number of reagent holders, each holder comprising: a tube containing liquid lysis reagents; a tube containing liquid wash reagents; a tube containing liquid neutralization reagents; and a tube containing liquid release reagents. Other numbers of tubes available in kit form include 12, 25, 30, 36, 48, 50, 60, and 100. Still other numbers are also permissible and consistent with the description herein.

Furthermore, in other embodiments of such a kit, the tube containing lyophilized microparticles can additionally contain particles of reagents selected from the group consisting of: proteinase-k; proteinase-k and mutanolysin; and proteinase-k, mutanolysin, and an internal control DNA. The additional enzymes are often used in cell-specific lysis applications.

### Conditions of DNA Binding and Elution

One factor to consider when assessing the efficacy of a DNA-capture material is the material's pKₐ. The pKₐ of an acid, HA, is an equilibrium constant for the equilibrium

HA [symbol] H⁺+A⁻,

given by pKₐ=−log₁₀Kₐ, where Kₐ=[H⁺][A⁻]/[HA]. It can be shown that, when the pH (=−log₁₀[H⁺]) of the solution is numerically equal to the pKₐ of the acid, the acid is 50% dissociated at equilibrium. Therefore, knowing the pKₐ of a material gives an indication of the pH, below which it is largely dissociated (in anion form), and above which it is largely unionized.

The pKₐ for an amino group is defined for its conjugate base, as follows: a protonated amine, R—NH₃⁺ is in dissociative equilibrium:

R—NH₃⁺ [symbol] H⁺+R—NH₂

and its pKₐ is given by −log₁₀Kₐ, where Kₐ=[H⁺][R—NH₂]/[R—NH₃⁺].

Because a nitrogen atom is trivalent, and due to the conditions of polymerization, each molecule of PEI can have a mixture of primary, secondary, and tertiary amine groups. Therefore, polyethylene molecules exhibit multiple pKₐ's over a range of values roughly consonant with the range of pKₐ's spanned by primary, secondary, and tertiary aliphatic amines, whose pKₐ's typically lie in the range 10-11, as evidenced by standard works in organic chemistry, e.g., Table 12.2 of Organic Chemistry, 2^{nd} Ed., Allinger, et al., Eds., Worth Publishers, Inc. (1976).

Accordingly, PEI typically has a pKₐ in the range greater than 9.0. Although a measured pKₐ value for the material supplied by Sigma-Aldrich is not available, since PEI contains a mixture of primary, secondary, and tertiary amine groups, it is likely to have a pKₐ the same range as other materials having such groups, i.e., in the range of 10-11.

PEI is effective as a binder for DNA in the processes described herein at least in part because the amine groups of the PEI have a pKₐ of between 10-11. Thus, at low pH it is typically positively charged—and may even carry multiple positive charges per molecule arising from protonations of the amine groups at pH's lower than its pKₐ—and is therefore able to bind strongly to polynucleotides such as DNA and RNA, which typically comprise polyanions (are predominantly negatively charged) in solution.

During the use of the PEI molecule in the processes described herein, the pH of the binding buffer (typically TRIS) used to lys cells at the same time as binding liberated DNA to the particles, is approximately 7-8. At this pH, all the amines (17 possible groups per PEI molecule, as available from Sigma) remain protonated (positively charged) and hence strongly attract negative charged DNA molecules to bind towards the beads.

PEI molecules are also advantageous because they are resistant to, e.g., are immune to, degradation by lytic enzymes, protease enzymes (e.g., mixtures of endo- and exo-proteases such as pronase that cleave peptide bonds), harsh chemicals such as detergents, and heat up to 95° C., and as such are able to bind DNA during the lysis process as well. Thus, cell lysis and DNA binding can be combined into a single (synchronous) step, thereby both saving time and at least one processing step. The strong binding of DNA molecules to PEI enables rapid washing of affinity beads coated in PEI to remove PCR inhibitors using a wash solution. The release of DNA from the affinity beads is effected by an elevation of temperature in the presence of a proprietary release reagent. As the quantity of beads used is very small (<1 μl), the DNA can be released in a final volume as low as 3 microliters. The released DNA is neutralized to a final volume of 5-50 microliters using a neutralization reagent and is now ready for downstream PCR.

Typically, the amount of sample introduced is about 500 microliters or less (e.g., about 250 microliters or less, about 100 microliters or less, about 50 microliters or less, about 25 microliters or less, about 10 microliters or less). In some embodiments, the amount of sample is about 2 microliters or less (e.g., about 0.5 microliters or less).

PEI gives excellent DNA recovery, based in part on its high binding capacity, and its high release efficiency. In general, the ratio of mass of particles to the mass of DNA retained by the particles is no more than about 25 or more (e.g., no more than about 20, no more than about 10). For example, in some embodiments, about 1 gram of particles retains about 100 milligrams of DNA; when used in smaller quantities, similar ratios can be obtained (e.g., a binding capacity of ˜100 μg of DNA/mg beads).

### Other Apparatus for DNA Capture

In other embodiments, the solid support can be configured as a retention member (e.g., porous member such as a column, filter, a porous membrane, a microporous filter, or a gel matrix, having multiple openings such as pores and/or channels, through which DNA passes) through which sample material (containing the DNA) must pass. Such a retention member may be formed of multiple surface-modified particles constrained into a suitable geometry. In some embodiments, the retention member comprises one or more filter membranes available from, for example, Osmonics, which are formed of polymers that may also be surface-modified and used to retain DNA. In some embodiments, a retention member is configured as a plurality of surfaces (e.g., walls or baffles) across which a sample passes. The walls or baffles are modified to retain DNA in preference to, e.g., PCR inhibitors. Such a retention member is typically used when the microparticles are non-magnetic.

As a sample solution moves through a processing region containing such a retention member (suitably modified to preferentially retain DNA), DNA is retained while the liquid and other solution components (e.g., inhibitors) are less retained (e.g., not retained) and exit the processing region. Typically, such a retention member retains at least about 50% of DNA molecules (at least about 75%, at least about 85%, at least about 90%) of the DNA molecules present in the sample that entered the processing region. The processing region is typically at a temperature of about 50° C. or less (e.g., 30° C. or less) during introduction of the sample. Processing can continue by washing the retention member with a wash solution to separate remaining inhibitors from DNA retained by the retention member.

In some embodiments, the sample preparation processes described herein are performed within a microfluidic device, such as a microfluidic cartridge configured to receive a sample, and to capture DNA molecules from the sample on a solid support contained within it. Exemplary microfluidic cartridges are described in U.S. Patent Application Publication No. 2006/0166233, and WO2008/061165, both of which are incorporated herein by reference. Such cartridges may include one or more actuators configured to move microdroplets of various liquid solutions within the cartridge, a chamber configured to lys cells in the sample, and one or more channels and associated valves configured to direct, disrupt, and divert liquid flow within the cartridge.

While sample preparation has been described as being a sequence of operations carried out in a single location, such as in a process tube or a microfluidic cartridge, other configurations can be used. For example, in some embodiments, the retention member carrying a DNA-affinity material can be removed from a region where DNA capture occurs for subsequent processing elsewhere. For example, the retention member may be contacted with a mixture comprising DNA and inhibitors in one location and then moved to another location at which the DNA are removed from the retention member.

### Other Advantages of the DNA Capture Material Described Herein

The extraction reagents and sample preparation processes described herein offer superior performances compared to currently available off-the-shelf kits for sample preparation. Advantages of the materials and methods herein include the following.

A streamlined sample preparation procedure having fewer steps (as few as six from raw sample to purified DNA) and utilizing fewer containers than other procedures.

Extraction control (cellular, plasmid or naked) DNA can also be included along with the affinity beads. An internal control DNA can be included with the lysis reagents so that the internal control DNA gets co-purified with the other DNA (such as the target DNA) present in the clinical sample, and gets eluted amongst the final released DNA. During amplification of the eluted DNA, the internal control DNA is also amplified, and can subsequently be detected using a separate fluorophore from the target DNA. This gives an extra confirmation that the sample prep process worked as required.

The description herein has included a characterization of properties and use of microparticles coated in PEI. It would be understood by one of ordinary skill in the art that other affinity molecules may suitably be used in the processes described herein, as described elsewhere (e.g., U.S. patent application publication 2006-0166233, incorporated herein by reference). Still other affinity molecules are described in U.S. patent application serial no. 12/______, filed on even date herewith and identified by attorney docket no. 19662-067001, and incorporated herein by reference.

### EXAMPLES

### Example 1

### Sample Preparation Process

The following six steps can be accomplished in as little as 15 minutes for a single sample, to 30 minutes for a batch of 12 samples, using a reagent kit as further described herein. The steps are also easily automated as in a system described in U.S. provisional patent application Ser. No. 60/959,437, filed Jul. 13, 2007 incorporated herein by reference. The steps are also shown, schematically, in FIG. 1, and described generally elsewhere herein.
1. Mix ˜500 μl of the clinical sample with 500 μl of lysis buffer and magnetic affinity beads, surface-bound with PEI. Kits for detecting gram positive bacteria include some lytic enzymes as well dissolved in the lysis buffer.
2. Incubate the mixture of sample, lysis buffer, and beads at a temperature between room temperature and 60° C. for 5-10 minutes, to lys the cells and bind the DNA to the affinity beads.
3. Separate the magnetic beads and remove as much of the supernatant solution as possible.
4. Wash the beads with a wash reagent.
5. Release the DNA from the beads by heating the beads for 3 minutes at 85° C. in the presence of as little as 3 microliters of release solution.
6. Remove the released DNA and neutralize the solution with a neutralization reagent, such as a Tris buffer, to create PCR-ready DNA.

### Example 2

### Application to a Wide Variety of Matrices

The procedures described herein work for a variety of sample matrices, including both clinical and non-clinical samples, as shown by the following non-exhaustive list:

Infectious Disease (Human)
Vaginal+Rectal Swab
Endocervical & Urethral Swabs
Genital Lesions
Pus
Nasal swab
Sputum
Whole Blood & Plasma
Urine
CSF
M4/UTM/Todd Hewitt broth

Cancer/Pharmacogenomics (Human Genomic DNA Prep)
Buccal Swab
Whole Blood

Veterinary/Food Testing
Ground Beef
Cheese

Agricultural Testing, such as for a genetically-modified crop product
Corn (seed)
Soy (seed/meal)
Maize

Environmental/Biodefense
Swab
Water

### Example 3

### Representative Results

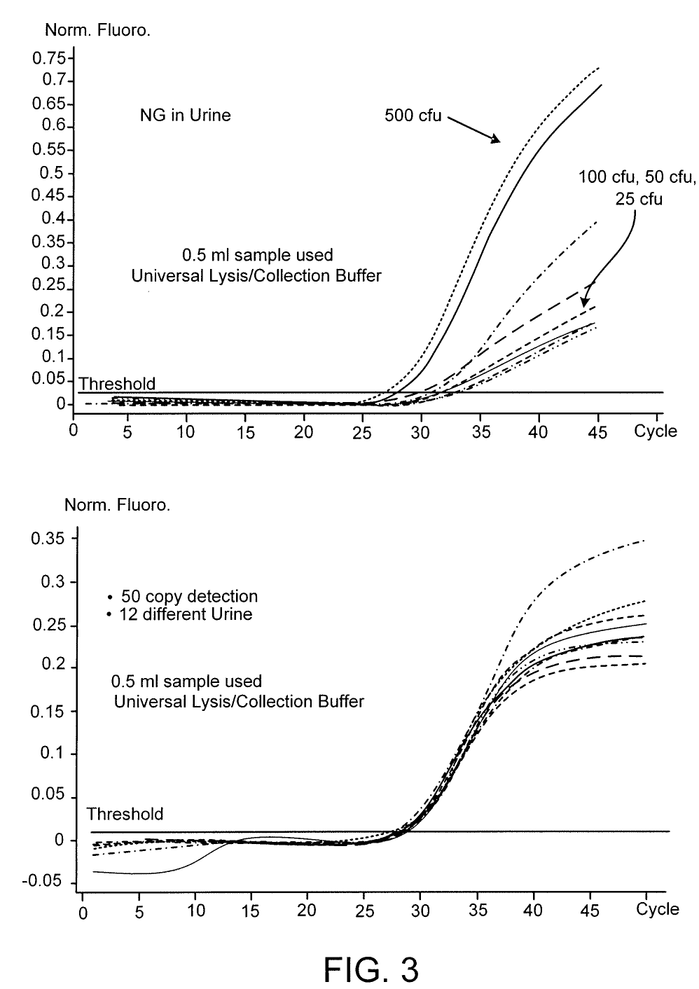

FIG. 3 shows the use of DNA extraction reagent, PEI, and a process as further described herein, to isolate and purify Neisseria Gonorrhoeae (NG) cells in urine at various concentrations of colony forming units (cfu). Both panels show PCR curves for various samples. The upper panel shows two PCR curves for samples having each of 500 cfu, 100 cfu, 50 cfu, and 25 cfu. In each case, a 0.5 ml sample size was used, in conjunction with a universal lysis/collection buffer. The lower panel shows PCR curves for 12 different 0.5 ml urine samples having 50 copies of NG in each.

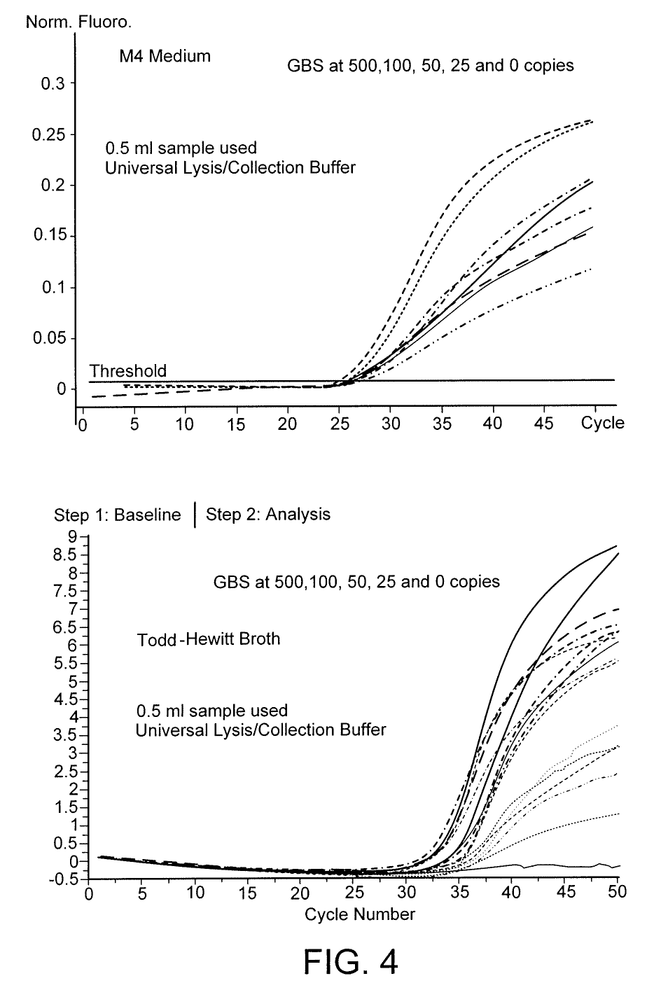

FIG. 4 shows PCR curves resulting from the use of DNA extraction reagent, PEI, and a process as further described herein, to isolate and purify Group B Streptococcus cells in two standard collection media M4 medium (upper panel) and Todd-Hewith broth (lower panel). The upper panel shows PCR curves for various concentrations, 500 cfu, 100 cfu, 50 cfu, and 25 cfu, with a negative control (0 cfu). This illustrates that the methods and materials are sensitive enough to detect concentrations of pathogen DNA as low as 25 cfu.

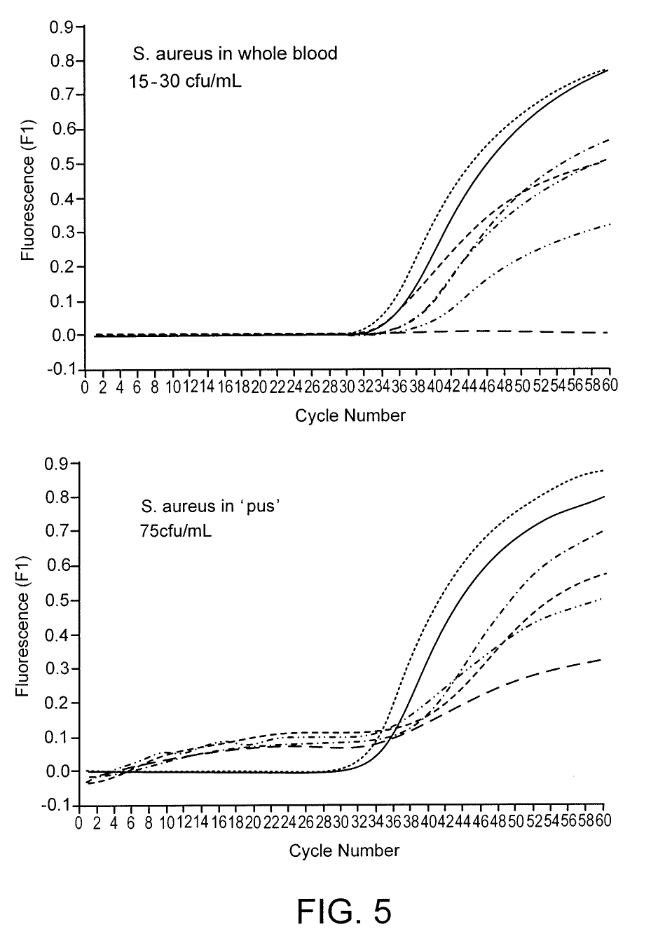

FIG. 5 shows the use of DNA extraction reagents, PEI, and a process as further described herein, to isolate and purify S. aureus cells in whole blood at concentrations in the range of 15-30 cfu per ml of blood (upper panel) and at a concentration of 75 cfu/ml pus (lower panel). The PCR curves illustrate detectable amplification of the pathogen DNA.

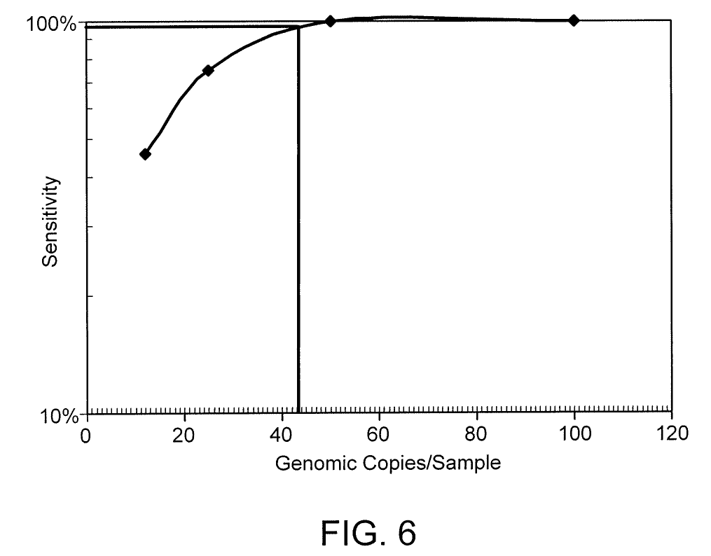

FIG. 6 shows illustrates the analytical sensitivity of the process: a plot of sensitivity against number of copies present is shown for Chlamydia trachomatis (CT) in clinical urine. Probit analysis reveals a LoD (limit of detection) of 43 copies/0.5 mL.

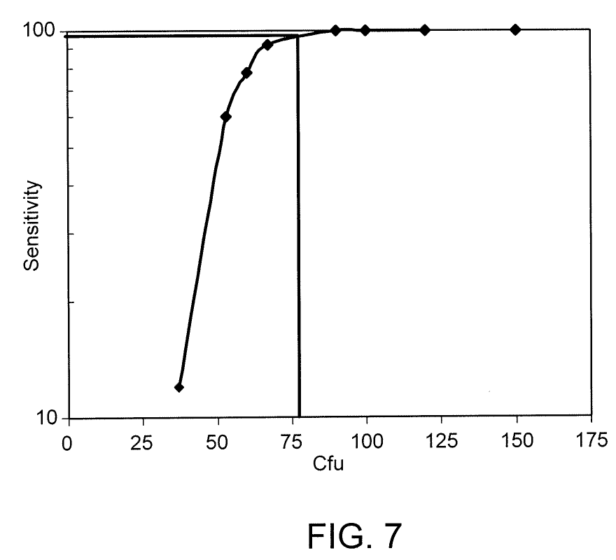

FIG. 7 shows illustrates the analytical sensitivity of the process: a plot of sensitivity against concentration of colony forming units is shown for GBS in mock swabs. Probit analysis reveals an LoD of 78 copies/0.5 mL.

### Example 5

### Quantitation of DNA Extraction Process

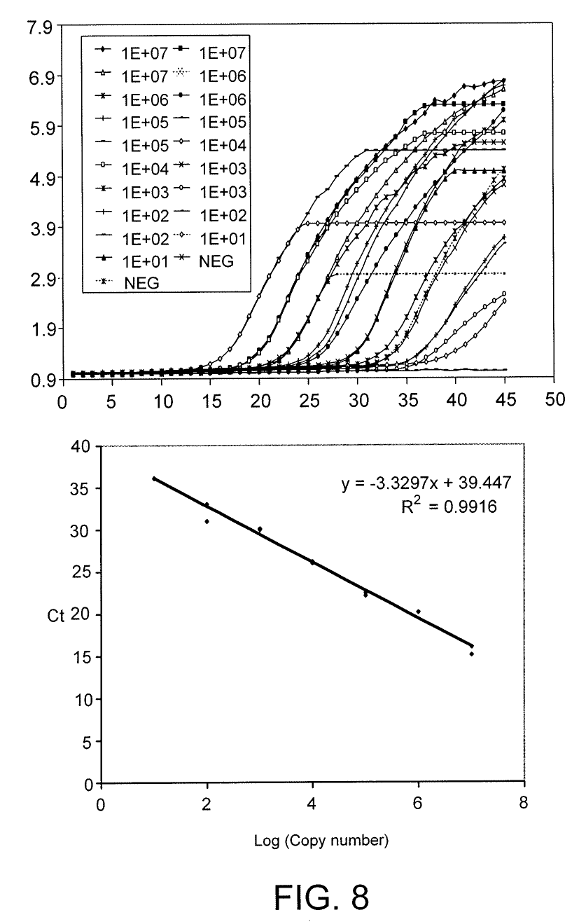

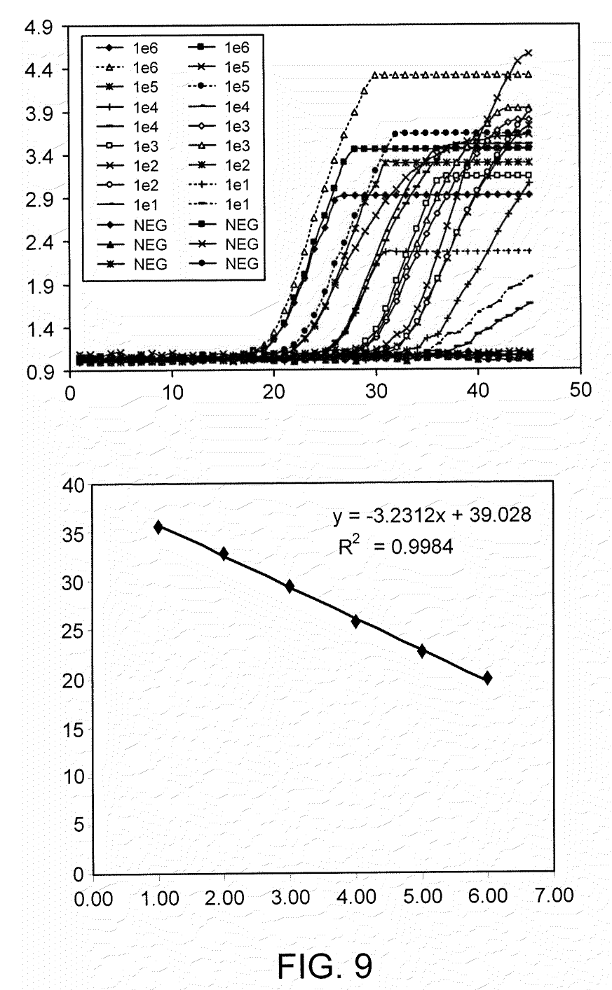

FIGS. 8 and 9 show how the processes herein can give quantitative DNA extraction over 1-7 log(dilution) units.

FIG. 8 shows quantitation in urine. PCR curves (left panel) for detection of NG in urine specimens for various numbers of copies give rise to a quantitative determination of the amount of DNA present. The right panel of FIG. 8 shows how, when Ct (the threshold cycle at which the fluorescence emerges from the baseline) is plotted against the log of the copy number, a straight line results, with a regression coefficient of 0.9912.

FIG. 9 shows quantitation in plasma. PCR curves (left panel) for detection of Chlamydia Trachomatis (CT) in plasma specimens for various numbers of copies give rise to a quantitative determination of the amount of DNA present. The right panel of FIG. 9 shows how, when threshold cycle (Ct) is plotted against Log (starting Concentration of target cell), a straight line results, with a regression coefficient of 0.9984.

### Example 6

### Exemplary Reagents for Use with Manual or Automated DNA Extraction Process

The reagents shown in the following table are typically utilized. Advantageously, the various sample preparation reagents are available in lyophilized form as well as liquid format. The lyophilized reagents (the enzyme pellets, and the Lyophilized magnetic affinity pellet) and methods of preparing the same are described in U.S. patent application publication 2007-0259348, incorporated herein by reference in its entirety). The magnetic DNA affinity microspheres can be in either liquid or lyophilized format.

Reagent Description

2× Lysis Buffer
Enzyme Pellets (ProK EM, ProK/RNAse EM, ProK/Mutano EM)
Reagent 1
Reagent 2
Reagent 3A
Reagent 3B
Reagent: Lyo Pellet, Affinity, Mag
Reagent: Microsphere, DNA Affinity, Mag (LIQUID)
UIPC

Certain of the reagents in the table have the following descriptions.

The exemplary “2× lysis buffer” has the following content. The designation “2×” means that the solution is made up at double the required concentration so that, when adding the sample solution, the concentration of lysis reagents in the resulting solution is correct.

| Constituent of 2X lysis buffer | For a 1 L volume | |
|---|---|---|
| Ultrapure water | 480 | mL |
| 1 M Tris-HCl pH 7.0 | 100 | mL |
| Triton X-100 | 20 | mL |
| 0.5M EDTA | 400 | mL |
| Boric Acid | 2.48 | g |
| Sodium Citrate | 11.76 | g |

It has been found that the presence of borate and citrate in the lysis solution helps to inhibit DNAses, in addition to the role of EDTA in this process.

“Reagent 1” is a wash solution, as follows.

| Constituent of Reagent 1 | For a 1 L volume | |
|---|---|---|
| 1M Tris-HCl pH 8.0 | 100 | μL |
| Sodium Azide | 200 | mg |
| Ultrapure water | 1,000 | ml |

Reagent 2 is a release solution, as follows.

| Constituent of Reagent 2 | For a 1 L volume |
|---|---|
| Ultrapure water | 600 mL |
| 0.1 N NaOH | 400 mL |

The storage of the release buffer over time should be regulated. Open containers of release solution (containing NaOH) decrease their pH over time to a value below 10, thereby impairing performance in the sample preparation processes described herein. It is therefore important that the release solution is maintained in closed air-tight containers to prevent—or minimize the likelihood of—this effect.

Reagent 3A is a neutralization solution for use in manual sample preparation.

| Constituent of Reagent 3A | For a 1 L volume | |
|---|---|---|
| 1.0M Tris-HCl pH 8.0 | 330.0 | mL |
| Sodium Azide | 200 | mg |
| ultrapure water | 670 | mL |

Reagent 3B is a neutralization solution for use in automatic sample preparation, such as described in U.S. provisional patent application Ser. No. 60/959,437, filed Jul. 13, 2007, incorporated herein by reference.

| Constituent of Reagent 3B | For a 1 L volume | |
|---|---|---|
| 1.0M Tris-HCl pH 8.0 | 200.0 | mL |
| Sodium Azide | 200 | mg |
| ultrapure water | 800 | mL |

UIPC, Universal Internal Process Control, is an optional component, comprising a DNA marker, typically present at a concentration of 1000 copies. It goes through the process, accompanying the amplification of target DNA and serves as a control: if it is not detected in amplified form, then there was some sort of processing error in the overall sample preparation process.

### Example 7

### Exemplary Manual DNA Extraction, and Preparation of PCR-Ready DNA, from Urine

The protocol described in this example illustrates a manual process, such as performed on an individual sample, in a laboratory, or on many samples in parallel, for example using sample holders and a rack, as described in U.S. provisional patent application Ser. No. 60/959,437, filed Jul. 13, 2007). The materials and reagents are as described elsewhere herein, such as in Example 4.

| Step | Action |
|---|---|
| 1 | Pipette 500 μl of specimen into a tube (1.7 ml DOT snap cap tube) containing 500 μl |
| | of 2X Lysis Buffer. |
| 2 | Pipette up and down twice, and then pipette entire amount into 3 ml syringe |
| 3 | Insert plunger into syringe, and filter contents into a tube (1.7 ml DOT snap cap tube) |
| | containing: |
| | 30 μl of magnetic beads or 2 lyophilized AB pellets. |
| | Apply pressure until all liquid is expelled, and foam starts to come out of filter (avoid |
| | getting foam into sample). |
| | Cap and invert reaction tube 5 times, or until beads are dispersed (or dissolved, in the |
| | case of lyophilized beads). |
| 4 | Incubate samples for 10 min. at room temperature (10.5 min. maximum). |
| 5 | Remove samples from water bath, and dry outsides of tubes with an absorbent wipe. |
| 6 | Place tubes on magnetic rack, and allow separation to proceed for 1 minute (1.5 min |
| | max). |
| 7 | Using a fresh pipette tip for each sample, carefully aspirate 1 ml of supernatant from |
| | each sample (without disturbing beads), using a 1 ml pipettor. |
| | Discard supernatant. Be sure to remove any liquid that remains in the tube cap. |
| 8 | After initial removal of supernatants from all samples, let liquid settle in tubes for 1 |
| | minute. |
| | Then, remove any remaining liquid using a fresh 1 ml pipette tip for each sample. |
| 9 | Place tubes in a non-magnetic tube rack, and add 100 μL of Reagent 1 to each tube |
| | using a 200 μl pipette tip. |
| | Pipette up and down 10 times, or until all magnetic beads are re-suspended, and no |
| | beads remain stuck to pipette tip. |
| 10 | Place tubes on magnetic rack for 30 seconds, allowing beads to separate. |
| 11 | Carefully aspirate supernatant from all samples using a 200 μl pipette tip. |
| | Discard supernatant. |
| | After initial removal of supernatants from all samples, let liquid settle in tubes for 1 |
| | minute. |
| | Then, using a fresh 200 μl tip for each sample, aspirate any remaining liquid left in |
| | the sample, and discard the liquid. |
| 12 | Place tubes in a non-magnetic tube rack, and add 8 μl of Reagent 2. |
| | Pipette up and down 10 times to resuspend magnetic beads. |
| 13 | Place samples in a heat block at 85° C. for 3 minutes. (3.5 min. max). |
| 14 | Remove samples from heat block, and place on magnetic rack for 30 seconds (1 min. |
| | max). |
| 15 | Keeping tubes on the magnetic rack, remove 7 μl of liquid, carefully avoiding |
| | magnetic beads on side of tube, and place in 0.65 ml DOT tube that has 3 μl of |
| | Reagent 3A pre-aliquotted into the tube. |
| 16 | Mix sample by pipetting up and down once. |
| | Sample is now ready for PCR. |

### Example 8

### Exemplary Manual Method for DNA Extraction and PCR Preparation from Plasma, CSF, and Culture Media

The protocol described in this example illustrates a manual process, such as performed on an individual sample, in a laboratory, or on many samples in parallel, for example using sample holders and a rack, as described in U.S. provisional patent application Ser. No. 60/959,437, filed Jul. 13, 2007). The materials and reagents are as described elsewhere herein, such as in Example 4.

| Step | Action |
|---|---|
| 1 | Pipette sample (500 μl of specimen, plus 500 μl of 2X lysis buffer into a 1.7 ml DOT |
| | snap cap tube containing 30 μl of magnetic beads or 2 lyophilized AB pellets. |
| | Cap and invert Reaction Tube 5 times, or until beads are dispersed (or dissolved, in |
| | the case of lyophilized beads). |
| 2 | Incubate at room temperature for 10 min. (10.5 min maximum). |
| 3 | Remove samples from water bath, and dry outsides of tubes with an absorbent wipe. |
| 4 | Place tubes on magnetic rack, and allow separation to proceed for 1 minute (1.5 min |
| | max). |
| 5 | Using a fresh pipette tip for each sample, carefully aspirate 1 ml of supernatant from |
| | each sample (without disturbing beads), using a 1 ml pipettor. |
| | Discard supernatant. |
| | Be sure to remove any liquid that remains in the tube cap. |
| 6 | After initial removal of supernatants from all samples, let liquid settle in tubes for 1 |
| | minute. |
| | Then, remove any remaining liquid using a fresh 1 ml pipette tip for each sample. |
| 7 | Place tubes in a non-magnetic tube rack, and add 100 μL of Reagent 1 to each tube |
| | using a 200 μl pipette tip. |
| | Pipette up and down 10 times, or until all magnetic beads are resuspended, and no |
| | beads remain stuck to pipette tip. |
| 8 | Place tubes on magnetic rack for 30 seconds, allowing beads to separate. |
| 9 | Carefully aspirate supernatant from all samples using a 200 μl pipette tip. |
| | Discard Supernatant. |
| | After initial removal of supernatants from all samples, let liquid settle in tubes for 1 |
| | minute. |
| | Then, using a fresh 200 μl tip for each sample, aspirate any remaining liquid left in |
| | the sample, and discard the liquid. |
| 10 | Place tubes in a non-magnetic tube rack, and add 8 μl of Reagent 2. |
| | Pipette up and down 10 times to resuspend magnetic beads. |
| 11 | Place samples in a heat block at 85° C. for 3 minutes. (3.5 min. max). |
| 12 | Remove samples from heat block, and place on magnetic rack for 30 seconds (1 min. |
| | max). |
| 13 | Keeping tubes on the magnetic rack, remove 7 μl of liquid, carefully avoiding |
| | magnetic beads on side of tube, and place in 0.65 ml DOT tube that has 3 μl of |
| | Reagent 3A pre-aliquotted into the tube. |
| 14 | Mix sample by pipetting up and down once. |
| | Sample is now ready for PCR. |

### Example 9

### Exemplary Manual Method of DNA Extraction and PCR Preparation from Vagina/Buccal Swabs

The protocol described in this example illustrates a manual process, such as performed on an individual sample, in a laboratory, or on many samples in parallel, for example using sample holders and a rack, as described in U.S. provisional patent application Ser. No. 60/959,437, filed Jul. 13, 2007). The materials and reagents are as described elsewhere herein, such as in Example 4.

| Step | Action |
|---|---|
| 1 | Pipette sample (500 μl of specimen, plus 500 μl of 2X lysis buffer into a 1.7 ml DOT |
| | snap cap tube containing 30 μl of magnetic beads or 2 lyophilized AB pellets. |
| | and 1 Proteinase K enzyme pellet. |
| | Cap and invert reaction tube 5 times, or until beads are dispersed (or dissolved, in the |
| | case of lyophilized beads). |
| 2 | Immediately place samples in a 60° C. water bath, and incubate for 10 min. (10.5 min |
| | maximum). |
| 3 | Remove samples from water bath, and dry outsides of tubes with an absorobent wipe. |
| 4 | Place tubes on magnetic rack, and allow separation to proceed for 1 minute (1.5 min. |
| | maximum). |
| 5 | Using a fresh pipette tip for each sample, carefully aspirate 1 ml of supernatant from |
| | each sample (without disturbing beads), using a 1 ml pipettor. |
| | Discard supernatant. |
| | Be sure to remove any liquid that remains in the tube cap. |
| 6 | After initial removal of supernatants from all samples, let liquid settle in tubes for 1 |
| | minute. |
| | Then, remove any remaining liquid using a fresh 1 ml pipette tip for each sample. |
| 7 | Place tubes in a non-magnetic tube rack, and add 100 μl of Reagent 1 to each tube |
| | using a 200 μl pipette tip. |
| | Pipette up and down 10 times, or until all magnetic beads are resuspended, and no |
| | beads remain stuck to pipette tip. |
| 8 | Place tubes on magnetic rack for 30 seconds, allowing beads to separate. |
| 9 | Carefully aspirate supernatant from all samples using a 200 μl pipette tip. |
| | Discard Supernatant. |
| | After initial removal of supernatants from all samples, let liquid settle in tubes for 1 |
| | minute. |
| | Then, using a fresh 200 μl tip for each sample, aspirate any remaining liquid left in |
| | the sample, and discard the liquid. |
| 10 | Place tubes in a non-magnetic tube rack, and add 8 μl of Reagent 2. |
| | Pipette up and down 10 times to resuspend magnetic beads. |
| 11 | Place samples in a heat block at 85° C. for 3 minutes. (3.5 min. max). |
| 12 | Remove samples from heat block, and place on magnetic rack for 30 seconds (1 min. |
| | max). |
| 13 | Keeping tubes on the magnetic rack, remove 7 μl of liquid, carefully avoiding |
| | magnetic beads on side of tube, and place in 0.65 ml DOT tube that has 3 μl of |
| | Reagent 3A pre-aliquotted into the tube. |
| 14 | Mix sample by pipetting up and down once. Sample is now ready for PCR. |

### Example 10

### Exemplary Automated Method of DNA Extraction and PCR Preparation

The protocol described in this example illustrates a manual process, such as performed on an individual sample, in a laboratory, or on many samples in parallel, for example using sample holders and a rack, as described in U.S. provisional patent application Ser. No. 60/959,437, filed Jul. 13, 2007). The materials and reagents are as described elsewhere herein, such as in Example 4.

| Step | Action |
|---|---|
| 1 | Start by adding up to 500 μl of clinical specimen (plasma, urine, cerebral spinal fluid, |
| | swabs, blood) to 500 μl of 2× lysis buffer in collection tube. |
| 2 | Filtration or centrifugation of sample is currently required for urine specimens: use 5 μM |
| | pore size filter. |
| 3 | Entire 1 ml sample is added to lysis tube on disposable strip for lysis/binding. |
| | Binding will proceed for 6-10 min at room temperature for all specimen types |
| | except for swab specimens, which will proceed for 6-10 min at 60° C.. |
| 4 | Transfer to the lysis tube requires two pipetting suck steps from the robot, each |
| | taking approximately half of the initial sample). |
| 5 | Following dispensing, pipettors are paused in place to allow liquid remaining in tip to |
| | stream down to bottom of tip (10-30 s), and liquid is expelled into lysis tube. |
| 6 | Temperature ramping of the heat block proceeds immediately upon starting the |
| | robotic procedure (initial heat is held at 50° C.). |
| 7 | Samples are incubated for 8.5 minutes at 60° C., after which time a magnet is raised to |
| | capture beads, and incubation then continues for an additional 1.5 min. |
| | (For certain organisms, e.g., for Group B Streptococcus, a protocol will be available |
| | for an 80° C. lysis.) |
| 8 | Supernatant is aspirated from tube, and 100-1,000 μl of Reagent 1, is added to the |
| | lysis tube that is still at 60° C.. |
| | Perform 10-20 suck/spits to resuspend beads (it may be preferable to suck/spit with |
| | 100-200 μl, and then dispense the remaining liquid; this may aid in dispersing |
| | beads). |
| 9 | Magnet is moved up for 30 seconds to capture beads, and supernatant is aspirated and |
| | discarded. |
| | Following liquid discard, pipettors are paused in place to allow liquid remaining in |
| | tip to stream down to bottom of tip, and liquid is expelled into waste tube. |
| 10 | Bring magnet down 5-20 mm from max up position to concentrate beads at bottom |
| | of lysis tube. |
| 11 | Add 10-20 μl of Reagent 2 and raise magnet up and down once to assist in |
| | dislodging beads from side of tube. |
| | Pipette 10-20 times to resuspend beads. |
| 12 | Start heater temperature ramp up to 85° C. (it takes approximately 1 minute to reach |
| | temperature). |
| 13 | Lower magnet completely, and heat at 85° C. for 3 min. |
| 14 | Raise magnet to maximum up position. |
| 15 | Aspirate 7-14 μl of liquid, and then aspirate 7-14 μl of Reagent 3B solution. |

### Example 11

### Exemplary Method of PCR Amplification Using a Rotor-Gene Real Time PCR Apparatus

The protocol described in this example illustrates a process of amplifying PCR-ready DNA, as prepared by methods described elsewhere herein.

| Step | Action |
|---|---|
| 1 | Add 10 μl of sample directly to PCR pellets that have been |
| | pre-placed in Rotor-Gene tubes (tubes should be |
| | held in a pre-chilled block). |
| | Pipette liquid up and down until PCR pellet is completely |
| | dissolved (pellets may initially stick to pipette tips, |
| | but they will eventually dissolve in liquid). |
| 2 | Cap tubes, and place in Rotor-Gene instrument. |
| 3 | Run Rotor-Gene program as follows: |
| | 50 cycles of 94° C. for 4 seconds, and 60° C. for 15 seconds, |
| | acquiring to FAM (Green) and ROX (Orange) for the 60° C. step |
| | only. |
| | Gain should be set at 5 for FAM/Sybr (Green) and 8 for ROX |
| | (Orange) |

### Example 12

### PEI as a Contaminant

Since PEI can itself, if detached from microparticles or other solid supports, act as a PCR inhibitor, the level of PEI contamination tolerated by PCR was determined.

PCR was performed on an automated diagnostic instrument (see, e.g., an instrument as described in U.S. patent application Ser. No. 11/985,577, incorporated herein by reference) using serial dilutions of 150 mM, 15 mM, 1.5 mM, 150 μM, 15 μM, and 1.5 μM of contaminating PEI. 150 mM is the concentration used for synthesis of beads per sample. It was found that PEI inhibits PCR on 500 GBS, 1,000° C. per microliter at concentrations equal to and above 15 μM.

Efforts to minimize PEI contamination during the sample preparation process by increasing the denature temperature in a PCR cycle (102° C. at 2 sec hold time) did not help. Also, throwing in non-specific plasmid DNA to bind to the PEI impurities and cause less target DNA to be similarly bound, did not help.

Based on calculating the concentration of GBS/IC primers (0.7 μM) and probes, they are present in the same level as 1.5 μM of PEI (for which PCR works). Adding more primers (specific or non-specific) than the total amount of PEI seems to make the PCR work for 15 μM PEI contamination.

### Example 13

### Exemplary Process for the Preparation of DNA Affinity Magnetic Microspheres

The procedure in this example provides a method appropriate for one batch of polyethylenimine coated magnetic microspheres, commonly referred to as Magnetic DNA-Affinity Microspheres. One batch consists of 5 individual reactions performed in 50 mL conical tubes with a final volume of 30-90 mL.

The following is a list of equipment utilized in the process.

Vortexer

Microcentrifuge

Magnetic Rack

1.7 mL microcentrifuge tubes

4-oz specimen containers

50 mL conical tubes

15 mL conical tubes

Centrifuge

pH meter

Pipettors

Pipettor tips

Ultrasonic dismembrator

dH₂O wash bottle

Task Wipers

Balance

Laboratory marker

Gloves and Labcoat

Orbital shaker

Labeling tape

Pipette filler

Serological pipettes

An operator performing this procedure must be competent with a microbalance, pipettors, pH meter, ultrasonic dismembrator and a microcentrifuge, and must know how to prepare buffers, and possess an excellent pipetting technique. Gloves, labcoat, and eye protection should be worn by the operator at all times. Ear protection must be worn during sonication steps. All solutions are prepared in a laminar flow hood.

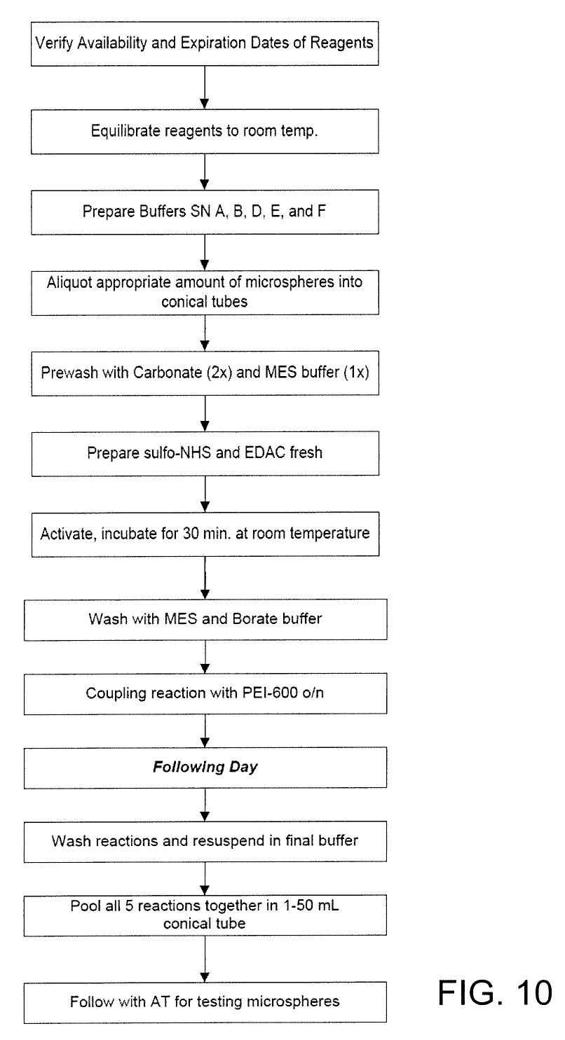

FIG. 10 shows a procedural flow chart outlining the exemplary procedure for making particles.

The following reagents are utilized herein, in the quantities indicated in the table.

| | For Five Reactions | | | |
|---|---|---|---|---|
| | For 30 mL | | For 90 mL | |
| Reagent | Total Vol. | | Total Vol. | |
| Carbonate buffer | 11 | ml | 33 | ml |
| Borate buffer | 46.4 | ml | 139.2 | ml |
| Buffer SN-C (0.5 M | 19 | ml | 57 | ml |
| MES buffer, pH 6.1) | | | | |
| 5 M NaCl | 22 | ml | 66 | ml |
| 10% Triton X-100 | 7.6 | ml | 22.8 | ml |
| EDAC | (approx. 62 | mg) | (approx. 186 | ml) |
| (N-(3- | | | | |
| dimethylaminopropyl)- | | | | |
| N′-ethyl-carbodiimide | | | | |
| hydrochloride) | | | | |
| PEI-600 (Polyethylenimine | 125 | mg | 375 | mg |
| 600, also known as | | | | |
| aziridine) | | | | |
| Sulfo-NHS | (approx. 375 | mg) | (approx. 1125 | mg) |
| (N- | | | | |
| Hydroxysulfosuccinimide, | | | | |
| sodium salt) | | | | |
| Carboxylic acid | 10 | ml | 30 | ml |
| modified magnetic | | | | |
| microparticles | | | | |
| Ultrapure water | 432 | ml | 1,296 | ml |

It has been found that the concentrations of EDAC and NHS are very important for successful synthesis of PEI-affinity beads.

Buffers can be prepared according to the following procedures, all of which are carried out in a laminar flow hood. Preliminary review of the reagents is as follows.

| Step | Action |
|---|---|
| 1 | Verify availability and check expiration dates of all solutions and |
| | reagents. |
| 2 | Visually inspect all stock solutions and reagents for precipitation or |
| | color change. Do not use if precipitation occurs or color changes. |
| 3 | Equilibrate all aliquoted stock solutions and reagents to RT. |
| | Take out EDAC and NHS from −20° C. and equilibrate |
| | to RT before use, this should take approximately one hour. |

Specific buffers are prepared as follows.

### Preparation of 110 or 330 mL Buffer SN-A (0.1M Carbonate Buffer, 0.15% Triton X-100)

| | | For | For |
|---|---|---|---|
| Step | Action | 30 mls | 90 mls |
| 1 | Label a 4-oz or 500 ml container with | | |
| | “Buffer SN-A”, date, and initials. | | |
| 2 | Using a serological pipette, add Carbonate | 11 | 33 |
| | Buffer to bottle. | | |
| 3 | With a P5000, add 10% Triton X-100 to bottle. | 1.65 | 4.95 |
| 4 | Using a graduated cylinder, add ultrapure water | 97.4 | 292.2 |
| | to bottle. | | |
| 5 | Mix well by inversion. | | |
| 6 | Check that the pH of solution is between | | |
| | 9.4 and 10.3. | | |
| 7 | Store at 4° C. during overnight incubation | | |
| | but discard after lot manufacture | | |
| | is complete. | | |

### Preparation of 140 or 420 mL Buffer SN-B (50 mM MES pH 6.1, 0.15% Triton X 100)

| | | For | For |
|---|---|---|---|
| Step | Action | 30 mls | 90 mls |
| 1 | Label a 4-oz or 500 ml container with | | |
| | “Buffer SN-B”, date, and initials. | | |
| 2 | Using a serological pipette add Buffer SN-C | 14 | 42 |
| | to bottle. | | |
| 3 | With a P5000, add 10% Triton X-100 to bottle. | 2.1 | 6.3 |
| 4 | Using a graduated cylinder, add ultrapure water | 124 | 372 |
| | to bottle. | | |
| 5 | Mix well by inversion. | | |
| 6 | Check pH of solution, which should be | | |
| | between 5.8 and 6.5 | | |
| 7 | Store at RT for the duration of lot manufacture, | | |
| | but discard after lot manufacture is | | |
| | complete. | | |

### Preparation of 110 or 330 mL Buffer SN-D (0.1M Borate Buffer, 0.15% Triton X-100)

| | | For | For |
|---|---|---|---|
| Step | Action | 30 mls | 90 mls |
| 1 | Label a 4-oz and 500 ml container with | | |
| | “Buffer SN-D”, date, and initials. | | |
| 2 | Using a serological pipette, add Borate Buffer | 22 | 66 |
| | to container. | | |
| 3 | With a P5000, add 10% Triton X-100 to | 1.65 | 4.95 |
| | container. | | |
| 4 | Using a graduated cylinder, add ultrapure water | 86.4 | 259.2 |
| | to container. | | |
| 5 | Mix well by inversion. | | |
| 6 | Check pH of solution, which should be | | |
| | between 8.7 and 9.3 | | |
| 7 | Store at 4° C. during overnight incubation but | | |
| | discard after lot manufacture is complete. | | |

### Preparation of 12 or 36 mL Buffer SN-E (0.1M Borate Buffer)

| | | For | For |
|---|---|---|---|
| Step | Action | 30 mls | 90 mls |
| 1 | Label a 15 mL or 50 mL conical tube with | | |
| | “Buffer SN-E”, date, and initials. | | |
| 2 | Using a serological pipette, add Borate | 2.4 | 7.2 |
| | Buffer to conical tube. | | |
| 3 | Using a serological pipette, add ultrapure | 9.6 | 28.8 |
| | water to tube. | | |
| 4 | Mix well by inversion. | | |
| 5 | Check pH of solution, which should be | | |
| | between 8.7 and 9.4. | | |
| 6 | Store at 4° C. during overnight incubation, | | |
| | but discard after lot manufacture is complete. | | |

### Preparation of 110 or 330 mL Buffer SN-F (0.1M Borate Buffer, 0.15% Triton X-100, 1M NaCl)

| | | For | For |
|---|---|---|---|
| Step | Action | 30 mls | 90 mls |
| 1 | Label a 4-oz or 500 ml container with | | |
| | “Buffer SN-F”, date, and initials. | | |
| 2 | Using a serological pipette, add 22 mL of | 22 | 66 |
| | Borate Buffer to conical tube. | | |
| 3 | With a serological pipette, add 22 mL 5M | 22 | 66 |
| | NaCl. | | |
| 4 | With a P5000, add 1.65 mL of 10% Triton | 1.65 | 4.95 |
| | X-100 to tube. | | |
| 5 | Using a graduated cylinder, add 64.4 mL | 64.4 | 193.2 |
| | ultrapure water. | | |
| 6 | Mix well by inversion. | | |
| 7 | Check pH of solution, which should be | | |
| | between 8.1 and 8.7. | | |
| 8 | Store at 4° C. during overnight incubation, | | |
| | but discard after lot manufacture is complete. | | |

### Synthesis of DNA-Affinity Microspheres

This is a two-day protocol. It is very important that the reagents EDAC and NHS be prepared freshly and used within 10 min. of preparation. The ambient humidity in the area where synthesis is carried out should be kept as low as is practical. Furthermore, the EDAC should be discarded and a fresh bottle opened after 5 uses regardless of how much is left.

Steps to be performed on Day 1, include the following.

| Step | Action |
|---|---|
| 1 | Calculate required amount of carboxylated microspheres. |
| | Divide 10 or 30 mL by % solids to calculate amount of |
| | microspheres needed per reaction. 5 reactions per set. |
| | Multiply this number by 5 to get the total amount of microspheres |
| | for the full set. |
| 2 | Vortex the vial containing the microspheres very well (for approx 1 |
| | minute). |
| 3 | Label 5-50 mL conical tubes with Lot number, date, and initials. |
| 4 | Pipette the appropriate amount of microspheres from squeeze tube |
| | into 50 mL conical tube. Repeat for all 5 reactions. |
| 5 | Place conical tubes onto magnetic rack and let sit until beads are |
| | fully captured by magnet. Remove supernatant carefully. |

| | |
|---|---|
| For 30 mls | For 90 mls |

Carbonate buffer washes

| | | | | | |
|---|---|---|---|---|---|
| 6 | Add Buffer SN-A to each tube and sonicate using | 10 | mls | 30 | mls |
| | ultrasonic dismembrator at power output of 12 for the | 10 | sec | 25 | sec |
| | appropriate time while rotating tube (ensure that probe is | | | | |
| | submerged). Clean probe with dH20 and wipe with an | | | | |
| | absorbent wipe before and after sonication. | | | | |
| | Place conical tubes onto magnetic rack and let sit until | | | | |
| | beads are fully captured by magnet. Remove supernatant | | | | |
| | carefully. | | | | |
| 7 | Repeat wash with Buffer SN-A. Add Buffer SN-A to each | 10 | mls | 30 | mls |
| | tube and sonicate using ultrasonic dismembrator at power | 10 | sec | 25 | sec |
| | output of 12 for the appropriate time while rotating tube | | | | |
| | (ensure that probe is submerged). Clean probe with dH₂O | | | | |
| | and wipe with an absorbent wipe before and after | | | | |
| | sonication. | | | | |
| | Place conical tubes onto magnetic rack and let sit until | | | | |
| | beads are fully captured by magnet. Remove supernatant | | | | |
| | carefully. | | | | |

MES buffer wash

| | | | | | |
|---|---|---|---|---|---|
| 8 | Add Buffer SN-B to each tube and sonicate using | 10 | mls | 30 | mls |
| | ultrasonic dismembrator at power output of 12 for the | 10 | sec | 25 | sec |
| | appropriate time while rotating tube (ensure that probe is | | | | |
| | submerged). Clean probe with dH₂O and wipe with an | | | | |
| | absorbent wipe before and after sonication. | | | | |
| | Place conical tubes onto magnetic rack and let sit until | | | | |
| | beads are fully captured by magnet. Remove supernatant | | | | |
| | carefully. | | | | |

Prepare sulfo-NHS

| | |
|---|---|
| 9 | Weigh out small amount of sulfo-NHS on weigh paper and multiply |
| | weight (in mg) by 20 to calculate μL of ultrapure water to add to make 50 mg/ml |
| | solution. |
| | For 30 mls, need 7.5 mL for 5 reactions (375 mg). |
| | For 90 mls, need 22.5 mL for 5 reactions (1125 mg). |
| | Add to 50 mL conical tube and mix well. |
| | Weight (mg) × 20 = μL ultrapure water needed. |
| | Add ultrapure water and vortex well to resuspend. |

| | |
|---|---|
| For 30 mls | For 90 mls |

Activation (Prepare EDAC in hood)

| | | | | | |
|---|---|---|---|---|---|
| 10 | Add reagents in the following order to each conical tube: | | | | |
| | (i) ddH₂O | 4910 | μL | 14730 | μL |
| | (ii) Buffer SN-C | 1000 | μL | 3000 | μL |
| | (iii) 50 mg/mL sulfo-NHS | 1500 | μL | 4500 | μL |
| | Sonicate using ultrasonic dismembrator at a power output | 10 | seconds | 25 | seconds |
| | of 12 for the appropriate time making sure that the probe | | | | |
| | is submerged at all times. | | | | |
| | Clean probe with dH₂O and wipe with an absorbent wipe | | | | |
| | before and after sonication. | | | | |

Immediately prepare 5 mg/ml EDAC in hood, as follows.

Prepare EDAC

| | |
|---|---|
| 11 | Weigh out small amount of EDAC onto weigh paper and multiply |
| | weight (in mg) by 200 to calculate μL of ultrapure water needed to make |
| | 5 mg/ml solution. |
| | For 30 ml total vol, need 12400 μl total (62 mg). |
| | For 90 ml total vol, need 37200 μl total (186 mg). |
| | Prepare in 50 mL conical tube. |
| | Weight (mg) × 200 μL ultrapure water to add. |
| | Vortex well after addition of ultrapure H₂O. |

| | | For 30 mls | | For 90 mls | |
|---|---|---|---|---|---|
| 12 | Add the following: | | | | |
| | (i) 10% Triton X-100 | 110 | μL | 330 | μL |
| | (ii) 5 mg/mL EDAC (add EDAC solution carefully; | 2480 | μL | 7440 | μL |
| | drop by drop while vortexing the solution at very | | | | |
| | low speed). | | | | |
| | Mix well by vortexing. | | | | |
| 13 | Secure tubes to orbital shaker with labeling tape and | 30 | mins | 90 | mins |
| | incubate at room temperature at setting 6 (or at setting | | | | |
| | where microspheres are mixing well). | | | | |
| 14 | After incubation, centrifuge for 5 or 15 min at maximum | 5 | mins | 15 | mins |
| | speed. Remove supernatant carefully but completely. | | | | |

MES buffer wash

| | | | | | |
|---|---|---|---|---|---|
| 15 | Add Buffer SN-B to each tube and sonicate using ultrasonic | 10 | mls | 30 | mls |
| | dismembrator at power output of 10 for the appropriate time | 10 | sec | 25 | sec |
| | while rotating tube (ensure that probe is submerged). Clean | | | | |
| | probe with dH₂O and wipe with kimwipe before and after | | | | |
| | sonication. | | | | |
| | Place conical tubes onto magnetic rack and let stand until | | | | |
| | beads are fully captured by magnet. Remove supernatant | | | | |
| | carefully. | | | | |

Borate buffer wash

| | | | | | |
|---|---|---|---|---|---|
| 16 | Add Buffer SN-D to each tube and sonicate using ultrasonic | 10 | mls | 30 | mls |
| | dismembrator at power output of 10 for the appropriate time | 10 | sec | 25 | sec |
| | while rotating tube (ensure that probe is submerged). | | | | |
| | Clean probe with dH₂O and wipe with absorbent wipe | | | | |
| | before and after sonication. | | | | |
| | Place conical tubes onto magnetic rack and let sit until | | | | |
| | beads are fully captured by magnet. Remove supernatant | | | | |
| | carefully. | | | | |

Prepare PEI

| | |
|---|---|
| 17 | Prepare 100 mg/mL PEI-600: Weigh out small amount of PEI-600 in 15 mL |
| | conical and multiply weight by 10 to determine amount of water to |
| | add to make 100 mg/mL. |
| | For 30 ml total volume, need 1250 μL total (125 mg). |
| | For 90 ml total volume, need 3750 μL total (375 mg). |
| | Vortex well to mix. |
| | Discard remaining 100 mg/mL PEI-600 after use. |

| | | For 30 mls | | For 90 mls | |
|---|---|---|---|---|---|
| 18 | Prepare coupling reaction: | | | | |
| | (i) Add Buffer SN-E | 2250 | μL | 6750 | μL |
| | Sonicate using ultrasonic dismembrator at power output 9 | 10 | sec | 25 | sec |
| | for the appropriate time making sure that the probe is | | | | |
| | submerged. Clean probe with dH₂O and wipe with | | | | |
| | absorbent wipe before and after sonication. | | | | |
| | (ii) Add 100 mg/mL PEI-600 (add solution carefully, | 250 | μL | 750 | μL |
| | drop by drop while vortexing the solution on low | | | | |
| | speed). | | | | |
| | (iii) Mix by vortexing. | | | | |

| | |
|---|---|
| 19 | Secure tube to orbital shaker with labeling tape. Incubate overnight at a |
| | setting of 6 at room temperature (or at setting where microspheres are |
| | mixing well). |
| 20 | Store buffers SN-B, D, and F at 4° C. overnight. Return NHS and EDAC |
| | stocks to −20° C. Return buffer SN-C to 4° C. Discard buffers SN-A |
| | and E. |

Steps to be performed on Day 2, include the following.

| | |
|---|---|
| 21 | After overnight incubation, remove buffers SN-B, D, and F from |
| | 4° C. and equilibrate to RT (approximately 1 hr). |
| 22 | Centrifuge tubes for 5 min(or 15 mins for 90 ml total vol) at |
| | maximum speed. Remove supernatant carefully but completely. |

| | |
|---|---|
| For | For |
| 30 mls | 90 mls |

Borate-NaCl washes

| | | | | | |
|---|---|---|---|---|---|
| 23 | Add Buffer SN-F to each tube and sonicate using | 10 | mL | 30 | mL |
| | ultrasonic dismembrator at power output of 12 for | 10 | sec | 25 | sec |
| | appropriate time while rotating tube (ensure that | | | | |
| | probe is submerged). Clean probe with dH₂O and | | | | |
| | wipe with absorbent wipe before and after | | | | |
| | sonication. | | | | |
| | Place conical tubes onto magnetic rack and let sit | | | | |
| | until beads are fully captured by magnet. Remove | | | | |
| | supernatant carefully. | | | | |
| 24 | Repeat Buffer SN-F wash. Add Buffer SN-F to | 10 | mL | 30 | mL |
| | each tube and sonicate using ultrasonic | 10 | sec | 25 | sec |
| | dismembrator at power output of 12 for the | | | | |
| | appropriate time while rotating tube (ensure that | | | | |
| | probe is submerged). Clean probe with dH₂O and | | | | |
| | wipe with kimwipe before and after sonication. | | | | |
| | Place conical tubes onto magnetic rack and let sit | | | | |
| | until beads are fully captured by magnet. Remove | | | | |
| | supernatant carefully. | | | | |

Borate wash

| | | | | | |
|---|---|---|---|---|---|
| 25 | Add Buffer SN-D to microspheres and sonicate | 10 | mL | 30 | mL |
| | using ultrasonic dismembrator at power output of | 10 | sec | 25 | sec |
| | 12 for 10 or 25 seconds while rotating tube | | | | |
| | (ensure that probe is submerged). Clean probe | | | | |
| | with dH2O and wipe with absorbent wipe before | | | | |
| | and after sonication. | | | | |
| | Place conical tubes onto magnetic rack and let sit | | | | |
| | until beads are fully captured by magnet. Remove | | | | |
| | supernatant carefully. | | | | |

Final Resuspension

| | | | | | |
|---|---|---|---|---|---|
| 26 | Resuspend each reaction in Buffer SN-B by | 6 | mL | 18 | mL |
| | sonication using ultrasonic dismembrator at power | 10 | sec | 25 | sec |
| | output of 12 for 10 or 25 seconds (ensure that | | | | |
| | probe is submerged). Clean probe with dH₂O and | | | | |
| | wipe with absorbent wipe before and after | | | | |
| | sonication. | | | | |

| | |
|---|---|
| 27 | Pool all 5 reactions together in 1 new 50 mL conical tube or 1 new |
| | 250 mL. Affix appropriate label to tube. |
| 28 | Discard Buffers SN-B, D, and F. Store Buffer SN-C at 4° C. and 5M |
| | NaCl at RT. |
| 29 | Store at 4° C. Stable for 1 month if stored appropriately. |

Agitation during coupling and activation is important to ensure all the magnetic beads get uniformly coated. The agitation also helps prevent the magnetic particles from settling at the bottom of the flask.

Three washes are performed after coupling of the PEI to the magnetic particles to ensure effective removal of unbound PEI.

The foregoing description is intended to illustrate various aspects of the instant technology. It is not intended that the examples presented herein limit the scope of the appended claims. The technology now being fully described, it will be apparent to one of ordinary skill in the art that many changes and modifications can be made thereto without departing from the spirit or scope of the appended claims.

1. A method for isolating DNA from a cell-containing sample, the method comprising:
contacting the sample with a lysis solution and a plurality of binding particles coated in polyethyleneimine, so that the DNA is liberated from the cells and becomes reversibly bound to the polyethyleneimine, thereby creating binding particles bound with DNA and a solution containing residual cellular matter;
compacting the binding particles bound with DNA;
removing the solution containing residual cellular matter;
washing the binding particles; and
releasing the DNA from the binding particles.

2. The method of claim 1, wherein the DNA has a size less than 7.5 Mbp.

3. The method of claim 1, wherein the polyethyleneimine is covalently bound to the surface of the plurality of binding particles.

4. The method of claim 1, wherein the polyethyleneimine has a molecular weight in the range 600-800 Da.

5. The method of claim 1, wherein the particles are made of a polymeric material selected from the group consisting of: polystyrene, latex polymers, polyacrylamide, and polyethylene oxide.

6. The method of claim 5, wherein the polymeric material is modified to provide one or more carboxylic acid groups, wherein the carboxylic acid groups provide an attachment point for the polyethyleneimine.

7. The method of claim 1, wherein the particles are magnetic.

8. The method of claim 7, wherein the particles have an average diameter of between about 0.5 microns and about 3 microns.

9. The method of claim 1, wherein the particles are non-magnetic and have an average diameter of between about 0.5 microns and about 10 microns.

10. The method of claim 1, wherein the particles are present in a density of about 10⁷-10⁹ particles per milliliter.

11. (canceled)

12. (canceled)

13. The method of claim 1, wherein the sample is any one of: vaginal-rectal swab, blood, urine, plasma, CSF, endocervical and urethral swab, pus, M4/UTM/Todd Hewitt broth, buccal swab, nasal swab, sputum, or water.

14. The method of claim 1, wherein the sample is a matrix comprising a genetically-modified crop product.

15. (canceled)

16. (canceled)

17. (canceled)

18. The method of claim 1, wherein the sample comprises cells of a pathogen, and DNA from the cells of the pathogen become bound to the polyethyeleneimine.

19. The method of claim 18, wherein the pathogen is Group B Streptococcus, and contacting the sample with the lysis solution and plurality of binding particles comprises incubating the sample with the solution and particles at 60° C. for 5-10 minutes.

20. The method of claim 18, wherein the pathogen is Chlamydia, and contacting the sample with the lysis solution and plurality of binding particles comprises incubating the sample with the solution and particles at 37° C. for 5-10 minutes.

21. (canceled)

22. (canceled)

23. (canceled)

24. (canceled)

25. (canceled)

26. (canceled)

27. (canceled)

28. (canceled)

29. The method of claim 1, wherein the method does not comprise centrifugation of the particles.

30. The method of claim 1, wherein the time required for completing the contacting, compacting, removing, washing, and releasing is between 10 and 30 minutes.

31. (canceled)

32. The method of claim 1, wherein the sample has a volume larger than the concentrated volume of the binding particles having the DNA bound thereto by a factor of at least about 10.

33. The method of claim 1, wherein the sample has a volume from 0.5 microliters to 3 milliliters, and the volume of the compacted particles is 2-3 μl.

34. The method of claim 1, wherein the ratio by weight of the DNA captured by the binding particles, to the binding particles prior to contact with the DNA, is 5-20%.

35. (canceled)

36. (canceled)

37. (canceled)

38. The method of claim 1, wherein the binding particles release 90% or more of the DNA bound thereto.

39. The method of claim 1, wherein the sample has a volume of 100 μl-1 ml, and the compacted beads occupy an effective volume of less than 2 microliters.

40. The method of claim 40, wherein after removal of the solution containing residual cellular matter, less than 10 microliters of solution is left along with the particles.

41. The method of claim 40, wherein the volume of wash buffer is less than 100 microliters.

42. The method of claim 40, wherein the DNA is released in a volume of less than 20 microliters of release solution.

43. The method of claim 1, wherein the particles are magnetic and the compacting comprises collecting the particles by applying an external magnetic field.

44. (canceled)

45. (canceled)

46. (canceled)

47. (canceled)

48. (canceled)

49. (canceled)

50. (canceled)

51. A kit, comprising:
a number of sealed tubes, each containing lysis buffer;
a tube containing lyophilized microparticles having polyethyeleneimine bound thereto;
a tube containing liquid wash reagents, sufficient to analyze the number of samples;
a tube containing liquid neutralization reagents, sufficient to analyze the number of samples; and
a tube containing liquid release reagents, sufficient to analyze the number of samples, wherein each component of the kit is stored in an air-tight container.

52. (canceled)

53. (canceled)

54. A kit, comprising:
a first air-tight pouch enclosing a number of tubes, each tube containing lyophilized microparticles having polyethyeleneimine bound thereto;
a second air-tight pouch enclosing a number of reagent holders, each holder comprising:
a tube containing liquid lysis reagents;
a tube containing liquid wash reagents;
a tube containing liquid neutralization reagents; and
a tube containing liquid release reagents.

55. (canceled)

56. (canceled)

57. (canceled)
